(19) United States
(12) Patent Application Publication
(10) Pub. No.: US 2017/0354787 A1
(43) Pub. Date: Dec. 14, 2017

(54) SAFETY SYRINGE WITH DOSE WINDOW

(71) Applicant: Brell Medical Innovations, LLC, Henrico, VA (US)

(72) Inventors: Jeffrey M. LEARY, Henrico, VA (US); Paul F. MEYERS, Fishers, IN (US)

(73) Assignee: Brell Medical Innovations, LLC, Henrico, VA (US)

(21) Appl. No.: 15/617,166

(22) Filed: Jun. 8, 2017

Related U.S. Application Data
(60) Provisional application No. 62/348,390, filed on Jun. 10, 2016.

(51) Int. Cl. A61M 5/315; A61M 5/32; A61M 5/00; A61M 5/31

(52) U.S. Cl. CPC A61M 5/31568; A61M 5/31528; A61M 5/002; A61M 5/3202; A61M 2230/201; A61M 2005/3126; A61M 2205/583

(57) ABSTRACT

An apparatus includes a syringe body, a plunger, a cap, and a dose gauge. The syringe body defines a volume configured to contain a medicament. The dose gauge is disposed about at least a portion of a side wall of the syringe body, and defines a window. The cap is coupled to a proximal end portion of the syringe body, and includes an engagement portion. The plunger has a distal end portion configured to move within the volume to convey the medicament. An engagement portion of the plunger is configured to interface with the engagement portion of the cap to prevent rotation of the plunger about a longitudinal axis of the plunger. An outer surface of the plunger includes a series of indicia. At least one of the indicium is visible through the window when the plunger is in a first position within the volume.

### CROSS-REFERENCE TO RELATED APPLICATIONS

This application claims benefit of priority to U.S. Provisional Application Ser. No. x62/348,390, entitled “Safety Syringe with Dosing Window,” filed Jun. 10, 2016, the disclosure of which is incorporated herein by reference in its entirety.

### BACKGROUND

The embodiments described herein relate to syringes and methods for administering a dose of medicament based on the subject's weight, height, age, or other characteristic.

Some known syringes for delivering dosages of a medicament include a syringe barrel with graduated markings and a plunger that is movable within the barrel to set the dosage and deliver the medicament. The graduated markings for such known syringes are often volumetric (i.e., they indicate the volume of the medicament within the syringe), and thus a user must calculate, convert, or otherwise determine the correct dose based on, among other things, the patient's weight, height and/or age. Additionally, the graduated markings are often very small, are sometimes provided in confusing or unfamiliar units of measure (e.g., teaspoons or milliliters), and require that the user align the plunger along the axis of motion to set the dosage. Moreover, certain medication regimens require that different amounts of the medicament be administered on different days of the treatment. Accordingly, using such known syringes and methods can result in an unacceptable level of medication error and/or “adverse drug events” because of improper dosing (delivering more or less of a drug than the prescribed amount), noncompliance with the regimen (missing a day, administering the improper amount for a given day), or the like.

Although such issues are prevalent with oral delivery of over-the-counter drugs, such as ibuprofen, Tylenol®, cough syrup, or the like, studies have shown that such issues also exist in hospital and clinical settings. For example, one study of adverse drug events at hospitals estimated that although a large number of adverse drug events occurred at the ordering stage, many occurred at the administering stage. Bates, D. W., et al., “Incidence of Adverse Drug Events and Potential Adverse Drug Events,” Journal of the American Medical Association, Jul. 5, 1995, Vol. 274, No. 1, pp. 29-30. Another study evaluating the ability of 100 registered nurses to calculate the correct dosage for oral, intramuscular, and intravenous drugs showed an average error rate of about 20 percent or higher, depending on the type of delivery mechanism. Bindler, et al., “Medication Calculation Ability of Registered Nurses,” Journal of Nursing Scholarship, 1991, 23:221-224.

One proposed solution to reduce medication error and/or “adverse drug events” is to deliver a predetermined dosage via a single-use prefilled syringe or cartridge. Although convenient for some drugs and/or therapeutic regimens, prefilled syringes are expensive, cumbersome to store, and impractical for many drugs (e.g., over-the-counter painkillers). Moreover, unless a caregiver maintains an inventory of prefilled syringes tailored to a variety of different patients (e.g., weight, age range, or the like), the use of prefilled syringes will still require that the user calculate, convert or otherwise determine the correct dose to be administered.

Other delivery devices for administering dosages, such as insulin pens, include dose-setting mechanisms that include rotatable caps or plungers, bulky multi-part container holders, and the like. Although these devices may be suitable for certain drugs and/or therapeutic regimens, such as chronic care situations (e.g., delivery of insulin), such known devices are impractical for many other drugs and/or therapeutic regimens. For example, administering an over-the-counter cold medicine via an expensive pen injector that requires adherence to specific instructions for use is impractical.

Thus, a need exists for improved methods and devices for easily and accurately delivering medicaments via a syringe.

### SUMMARY

Syringes for delivering a dose of medicament are described herein. In some embodiments, an apparatus includes a syringe body, a plunger, a cap, and a dose gauge. The syringe body defines a volume configured to contain a medicament. The dose gauge is disposed about at least a portion of a side wall of the syringe body, and defines a window. The cap is coupled to a proximal end portion of the syringe body, and includes an engagement portion. The plunger has a distal end portion configured to move within the volume of the syringe body to convey the medicament. An engagement portion of the plunger is configured to interface with the engagement portion of the cap to prevent rotation of the plunger about a longitudinal axis of the plunger. An outer surface of the plunger includes a series of indicia. At least one of the indicium is visible through the window of the dose gauge when the plunger is in a first position within the volume of the syringe body.

### BRIEF DESCRIPTION OF THE DRAWINGS

FIG. 1 is an exploded view schematic illustration of a syringe assembly, according to an embodiment.

FIG. 2 is a top view of a portion of the syringe assembly shown in FIG. 1, taken along the line X-X in FIG. 1.

FIGS. 3-4 are schematic illustrations of the syringe assembly shown in FIG. 1, in a first and second configuration, respectively.

FIG. 5 is an exploded view schematic illustration of a syringe assembly, according to an embodiment.

FIG. 6 is a cross-sectional view of a portion of the syringe assembly shown in FIG. 5, taken along the line X-X in FIG. 5.

FIG. 7 is a front view schematic illustrations of the syringe assembly shown in FIG. 5.

FIGS. 8 and 9 are a front perspective view and a rear perspective view, respectively, of a syringe assembly, according to an embodiment.

FIGS. 10 and 11 are a front exploded view and a rear exploded view, respectively, of the syringe assembly shown in FIGS. 8 and 9.

FIG. 12 is a perspective view of a syringe body of the syringe assembly shown in FIGS. 8 and 9.

FIGS. 13 and 14 are a front perspective view and a rear perspective view, respectively, of the dose gauge of the syringe assembly shown in FIGS. 8 and 9.

FIG. 15 is a perspective view of the elastomeric member of the syringe assembly shown in FIGS. 8 and 9.

FIGS. 16 and 17 are a front perspective view and a rear perspective view, respectively, of the plunger of the syringe assembly shown in FIGS. 8 and 9.

FIGS. 18-20 are a front perspective view, a bottom perspective view, and a top view, respectively, of the cap of the syringe assembly shown in FIGS. 8 and 9.

FIG. 21 is a front view of the syringe assembly shown in FIGS. 8 and 9 with the plunger in a first position.

FIGS. 22 and 23 are a front perspective view and a rear perspective view, respectively, of a syringe assembly, according to an embodiment.

FIGS. 24 and 25 are a front perspective view and a rear perspective view, respectively, of the dose gauge of the syringe assembly shown in FIGS. 22 and 23.

FIG. 26 is a flow chart of a method of assembling a syringe assembly, according to an embodiment.

FIG. 27 is a front view of a package for a syringe assembly, according to an embodiment.

### DETAILED DESCRIPTION

Syringes for delivering a dose of medicament are described herein. In some embodiments, an apparatus includes a syringe body, a plunger, a cap, and a dose gauge. The syringe body defines a volume configured to contain a medicament. The dose gauge is disposed about at least a portion of a side wall of the syringe body, and defines a window. The cap is coupled to a proximal end portion of the syringe body, and includes an engagement portion. The plunger has a distal end portion configured to move within the volume of the syringe body to convey the medicament. An engagement portion of the plunger is configured to interface with the engagement portion of the cap to prevent rotation of the plunger about a longitudinal axis of the plunger. An outer surface of the plunger includes a series of indicia. At least one of the indicium is visible through the window of the dose gauge when the plunger is in a first position within the volume of the syringe body.

In some embodiments, an apparatus includes a syringe body and a plunger. The syringe body defines a volume configured to contain a medicament. A distal end portion of the syringe body includes a delivery tip. A proximal end portion of the syringe body defines a first opening through which the volume can be accessed. The proximal end portion of the syringe body includes a cap having a distal surface and a proximal surface, the proximal surface extending above the first opening. The cap defines a second opening between the proximal surface and the distal surface. A central axis of the second opening is nonparallel to a central axis of the first opening. The syringe body further includes a dose gauge disposed about a side wall of the syringe body, and that defines a window. The plunger has a distal end portion configured to move within the volume of the syringe body from a first position to a second position to convey the medicament. An outer surface of the plunger includes a series of indicia, with at least one indicium from the series being visible through the window of the dose gauge when the plunger is in the first position. The second opening of the cap is sized such that the distal end portion of the plunger can be removed from the volume of the syringe body via the second opening.

In some embodiments, an apparatus includes a syringe body, a plunger, a dose gauge, and a cap. The syringe body defines a volume configured to contain a medicament. A proximal end portion of the syringe body includes a flange, and a distal end portion of the syringe body includes a delivery tip. The plunger has a distal end portion configured to move within the volume of the syringe body to convey the medicament. An outer surface of the plunger includes a series of indicia. The dose gauge is coupled to the flange of the syringe body and defines a window. When the plunger moves within the syringe body, at least one indicium from the series of indicia is visible through the window of the dose gauge. The cap is coupled to the flange of the dose gauge and/or the flange of the syringe body, and has an engagement portion that is configured to interface with an engagement portion of the plunger to limit rotation of the plunger about a longitudinal axis of the plunger.

In some embodiments, an apparatus includes a syringe body, a plunger, and a dose gauge. The syringe body defines a volume configured to contain a medicament. A proximal end portion of the syringe body includes an engagement portion and a flange, and a distal end portion of the syringe body includes a delivery tip. The plunger has a distal end portion configured to move within the volume of the syringe body to convey the medicament. An engagement portion of the plunger is configured to interface with the engagement portion of the syringe body to limit rotation of the plunger about a longitudinal axis of the plunger. An outer surface of the plunger includes a series of indicia. The dose gauge is coupled to the flange of the syringe body and the dose gauge defines a window. When the plunger moves within the syringe body, at least one indicium from the series of indicia can be seen through the window of the dose gauge.

In some embodiments, a plunger can include a series of non-volumetric indicia. The non-volumetric indicia can be associated with a patient's weight, height, age and/or any other suitable dose-setting characteristic. In some embodiments, the series of indicia can be arranged coaxially along a longitudinal axis of the plunger.

In some embodiments, a kit includes a medicament container and a syringe assembly. The medicament container contains at least one dose of a medicament and defines an opening. The syringe assembly includes a syringe body, a plunger, a dose gauge, and a cap. The syringe body defines a volume configured to contain a medicament. A proximal end portion of the syringe body includes a flange and a distal end portion of the syringe body includes a delivery tip. The plunger has a distal end portion configured to move within the volume of the syringe body to convey the medicament. An outer surface of the plunger includes a series of indicia. The dose gauge is coupled to the flange of the syringe body and the dose gauge defines a window. When the plunger moves within the syringe body, the indicia of the plunger can be seen through the window of the dose gauge. The cap is coupled to the flange of the dose gauge and the flange of the syringe body. The cap has an engagement portion that is configured to interface with an engagement portion of the plunger to limit rotation of the plunger about a longitudinal axis of the plunger.

In some embodiments, the kit further includes a lock member removably coupled to the proximal end portion of the syringe body and a proximal end portion of the plunger. The lock member is configured to limit movement of the distal end portion of the plunger within the syringe body. The lock member includes an instruction indicia, such as, for example, drug labeling, warnings, or the like.

In some embodiments, a method includes inserting a delivery tip of a syringe body, which defines an internal volume therein, into a medicament container. The medicament container is then turned upside down. A plunger is moved in a proximal direction within the internal volume of the syringe body to convey the medicament from the medicament container into the internal volume of the syringe body. The moving is performed until a dose indicium from a plurality of dose indicia on the plunger is visible through a window of a dose gauge. For example, the moving is performed until the correct weight range of a patient is visible in the window of the dose gauge. The plunger includes an engagement portion configured to interface with an engagement portion of the syringe body or a cap to limit rotation of the plunger about a longitudinal axis of the plunger during the moving. The delivery tip of the syringe body is then removed from the medicament container and placed in the mouth of a patient. The plunger is then moved in a distal direction within the internal volume of the syringe body to convey the medicament from the internal volume via the delivery tip into the patient's mouth.

In some embodiments, a method of assembling a syringe includes coupling a dose gauge about a side wall of a syringe body. The syringe body defines a volume configured to contain a medicament, and has a distal end portion including a delivery tip. The dose gauge defines a window. A cap is coupled to a proximal end portion of the syringe body. The cap includes a distal surface and a proximal surface. The cap defines a lateral opening between the proximal surface and the distal surface. A distal end portion of a plunger is disposed into the volume via the lateral opening. An outer surface of the plunger includes a series of indicia. At least one indicium from the series is visible through the window of the dose gauge when the plunger is moved in a distal direction within the volume.

The term “about” when used in connection with a referenced numeric indication means the referenced numeric indication plus or minus up to 10 percent of that referenced numeric indication. For example, “about 100” means from 90 to 110.

As used in this specification, the words “proximal” and “distal” refer to direction closer to and away from, respectively, an operator of the medical device. Thus, for example, the end of the syringe assembly contacting the patient's body (e.g., being inserted into the mouth, supporting a needle, or the like) would be the distal end of the syringe assembly, while the end opposite the distal end would be the proximal end of the syringe assembly.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation, in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description. As another example, two structures described herein as being “substantially parallel” is intended to convey that, although a parallel geometric relationship is desirable, some non-parallelism can occur in a “substantially parallel” arrangement. As yet another example, a structure defining a volume that is “substantially 0.50 milliliters (mL)” is intended to convey that, while the recited volume is desirable, some tolerances can occur when the volume is “substantially” the recited volume (e.g., 0.50 mL). Such tolerances can result from manufacturing tolerances, measurement tolerances, and/or other practical considerations (such as, for example, minute imperfections, age of a structure so defined, a pressure or a force exerted within a system, and/or the like). As described above, a suitable tolerance can be, for example, of ±10% of the stated geometric construction, numerical value, and/or range. Furthermore, although a numerical value modified by the term “substantially” can allow for and/or otherwise encompass a tolerance of the stated numerical value, it is not intended to exclude the exact numerical value stated.

As used herein, the term “set” can refer to multiple features or a singular feature with multiple parts. For example, when referring to set of walls, the set of walls can be considered as one wall with multiple portions, or the set of walls can be considered as multiple, distinct walls. Thus, a monolithically-constructed item can include a set of walls. Such a set of walls can include, for example, multiple portions that are either continuous or discontinuous from each other. A set of walls can also be fabricated from multiple items that are produced separately and are later joined together (e.g., via a weld, an adhesive, or any suitable method).

As used in this specification, the term “medicament” includes any constituent of a therapeutic substance. A medicament can include such constituents regardless of their state of matter (e.g., solid, liquid or gas). Moreover, a medicament can include the multiple constituents that can be included in a therapeutic substance in a mixed state, in an unmixed state and/or in a partially mixed state. A medicament can include both the active constituents and inert constituents of a therapeutic substance. Accordingly, as used herein, a medicament can include non-active constituents such as, water, colorant or the like.

The term “fluid-tight” is understood to encompass hermetic sealing (i.e., a seal that is gas-impervious) as well as a seal that is only liquid-impervious. The term “substantially” when used in connection with “fluid-tight,” “gas-impervious,” and/or “liquid-impervious” is intended to convey that, while total fluid imperviousness is desirable, some minimal leakage due to manufacturing tolerances, or other practical considerations (such as, for example, the pressure applied to the seal and/or within the fluid), can occur even in a “substantially fluid- tight” seal. Thus, a “substantially fluid-tight” seal includes a seal that prevents the passage of a fluid (including gases, liquids and/or slurries) therethrough when the seal is maintained at pressures of less than about 5 psig. Any residual fluid layer that may be present on a portion of a wall of a container after component defining a “substantially-fluid tight” seal are moved past the portion of the wall are not considered as leakage.

The term “opaque” is understood to include structures (such as portions of a syringe body) that are not transparent and/or that do not permit an object to be clearly or distinctly seen through the structure. The term “opaque” or “substantially opaque” or “semi-opaque” when used in connection with the description of a side wall of a syringe body or any other structure described herein is intended to convey that objects cannot be clearly seen through the side wall. A side wall (or portion thereof) described as being “opaque” or “substantially opaque” or “semi-opaque” is understood to include structures that may have a blocking color, or that may not have a color, but that are otherwise hazy, blurry, smeared, textured or the like.

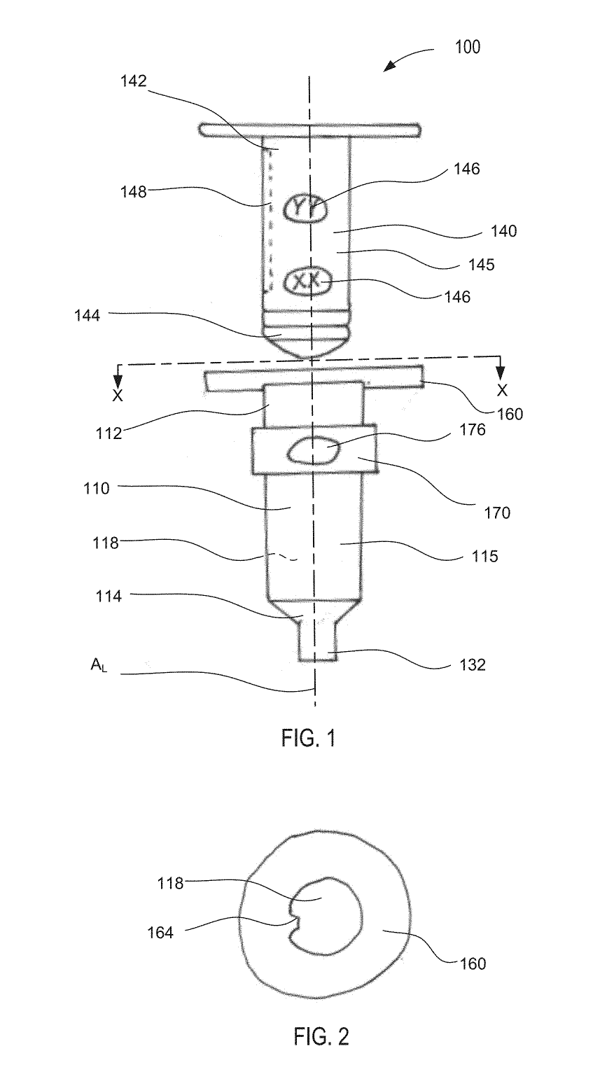

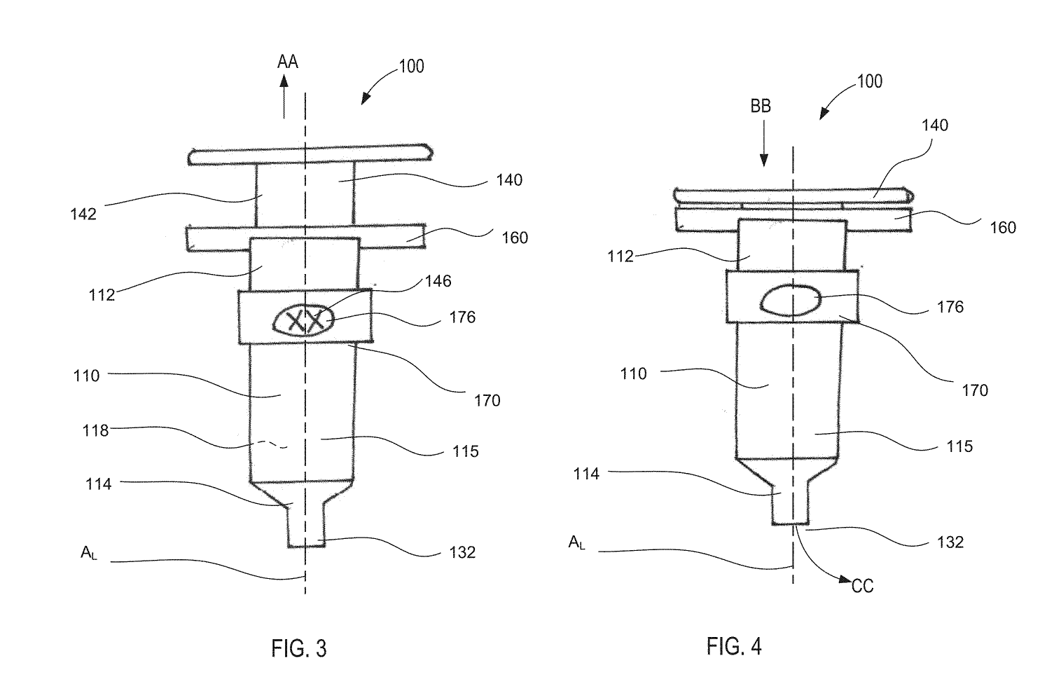

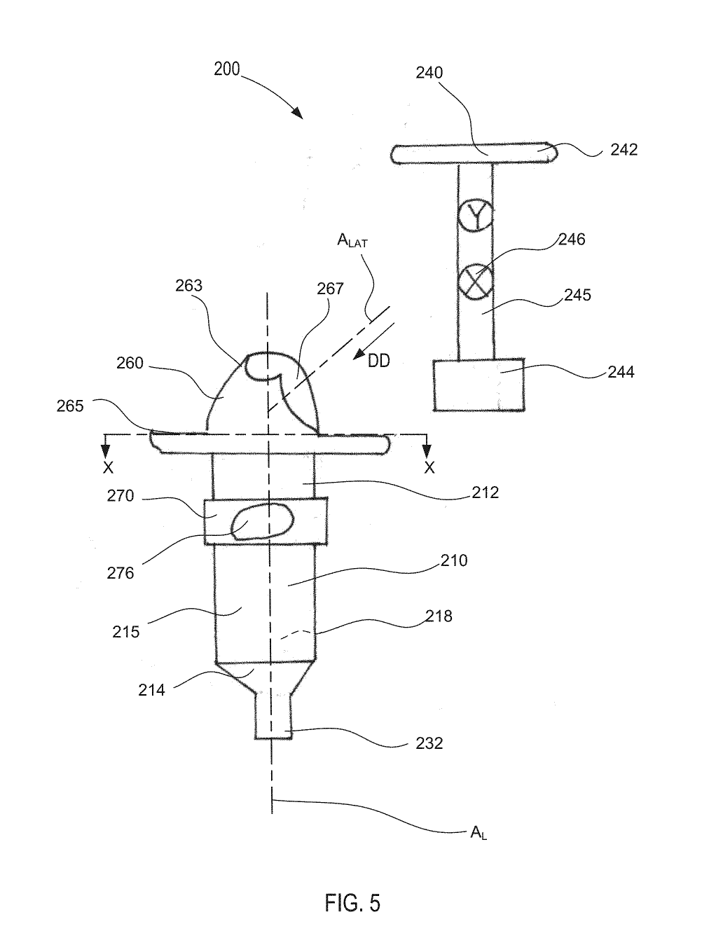

FIGS. 1-4 are schematic illustrations of a syringe assembly 100, according to an embodiment that includes a syringe body 110, a dose gauge 170, a cap 160, and a plunger 140. FIG. 1 shows an exploded view of the syringe assembly 100 to illustrate the syringe body 110 and the plunger 140. FIG. 2 shows a top view of the cap 160 and the syringe body 110 taken along the line X-X in FIG. 1. FIGS. 3-4 show the syringe assembly 100 in a first and a second configuration, respectively. The syringe body 110 includes a side wall 115, and defines an interior volume 118 within which a medicament can be contained. The syringe body 110 includes a proximal end portion 112, a distal end portion 114, and defines a longitudinal axis A_{L}. When the plunger 140 is disposed within the syringe body 110, the longitudinal axis A_{L} of the syringe body 110 is coaxial with the longitudinal axis A_{L} of the plunger 140. Thus, the longitudinal axis A_{L} shown and described can be considered the longitudinal axis for the syringe body 110, the plunger 140, or both the syringe body 110 and the plunger 140.

The distal end portion 114 of the syringe body 110 has a delivery tip 132. The delivery tip 132 can be any suitable tip or member through which the medicament can be conveyed either into or out of the internal volume 118 of the syringe body 110. In some embodiments, the delivery tip 132 can be a protrusion extending from the syringe body 110 that can be received within a medicament container (e.g., bottle, vial or the like) and that can also deliver the medicament orally to the patient. In other embodiments, the delivery tip 132 can a tapered fitting (such as a Luer fitting) that is adapted to couple the distal end portion 114 of the syringe body 110 to a needle (not shown).

In some embodiments, the syringe body 110 (and any of the syringe bodies described herein) can be constructed from a clear material (e.g., plastic or glass), and can include markings or graduated lines on the outer surface of the side wall 115. In some embodiments, the syringe body 110 (and any of the syringe bodies described herein) can include an opaque label (not shown) that defines one or more transparent windows through which a user can view the medicament or any other contents (e.g., the plunger 140) within the internal volume 118. In other embodiments, the syringe body 110 (and any of the syringe bodies described herein) can include a monolithically constructed opaque or semi-opaque portion. Such portions can include a light blocking color, or can be devoid of color, but can otherwise by hazy, blurred or textured to prevent medicament or other contents within the internal volume 118 from being clearly seen.

The dose gauge 170 is coupled about at least a portion of the side wall 115 of the syringe body 110, and defines a window 176. In particular, the dose gauge 170 is coupled about a transparent portion of the side wall 115 so that portions of the plunger 140 (i.e., the indicia 146) within the syringe body 110 can be seen through the window 176. In this manner, as described in more detail below, the dose gauge 170 and the plunger 140 can provide a visual indication of the dosage of medicament drawn into the syringe body 110. In some embodiments, a portion of the dose gauge 170 surrounding the window 176 is opaque, or otherwise includes a visible frame (not shown) that surrounds the window 176. In this manner, during use, the indicia 146 displayed within the window 176 are clearly accentuated to the user. Moreover, in some embodiments, an outer surface of the dose gauge 170 can include instructions or indicia (not shown) associated with the medicament, dose, and/or treatment regimen. Such instructions or indicia can include, for example, the characteristic of the patient to be considered when setting the dosage (e.g., “child's weight”), alignment marks or arrows, identification of the regimen (e.g., “day 2”), and/or the drug name.

The window 176 can be a portion of the dose gauge that is devoid of material. In other embodiments, however, the window 176 can include a transparent material through which a user can view the indicia 146. Moreover, the window 176 can be of any suitable size and/or shape.

Thus, although shown as being elliptical, in other embodiments, the window 176 can be rectangular. Moreover, although the window 176 is shown as being sized such that only one indicium 146 from the series of indicia is visible through the window at a time, in other embodiments, the dose gauge 170 (or any of the dose gauges described herein) can include a window through which multiple indicia can be viewed at a time. In yet other embodiments, the dose gauge 170 (or any of the dose gauges described herein) can include more than one window.

Although the dose gauge 170 is shown as extending only a portion of the length of the syringe body 110, in other embodiments, the dose gauge 170 (or any of the dose gauges described herein) can have a length that is substantially the same as the syringe body 110 (i.e., can extend substantially the full length of the syringe body 110). Moreover, the dose gauge 170 (or any of the dose gauges described herein) can extend around any portion of the circumference of the syringe body 110. For example, in some embodiments, the dose gauge 170 can extend around the full circumference of the syringe body 110. In this manner, the window 176 can be disposed about one portion of the syringe body 110 and other portions (e.g., opaque portions) of the dose gauge 170 can be disposed about another portion of the syringe body. By covering certain portions of the syringe body 110, the user can more easily identify the visual dose indication. In other embodiments, however, the dose gauge 170 can extend around only a portion of the circumference of the syringe body 110. For example, in some embodiments, the dose gauge 170 can extend around about three quarters of the circumference (about 270 degrees). In other embodiments, the dose gauge 170 can extend around about one half to three quarters of the circumference (from about 180 degrees to about 270 degrees). In other embodiments, the dose gauge 170 can extend around about one quarter to one half of the circumference (from about 90 degrees to about 180 degrees). In this manner, the window 176 can be disposed about one portion of the syringe body 110 and other portions of the syringe body 110 can be exposed. By exposing certain portion (e.g., the “back side) of the syringe body 110, the user can visually inspect the medicament in the syringe body 110 for air bubbles, the correct color, and the like.

The dose gauge 170 can be coupled to the syringe body 110 in any suitable manner. In some embodiments, the dose gauge 170 can be bonded to the side wall 115 of the syringe body 110 (e.g., via an adhesive, a weld joint or the like). In other embodiments, the dose gauge 170 can be coupled to the cap 160 or a flange (not shown) of the syringe body 110 to couple the dose gauge 170 about the side wall 115. As discussed below, by indexing the dose gauge 170 to the cap 160, the dose gauge 170 can be maintained in a fixed location along the longitudinal axis A_{L} of the syringe body 110, thereby producing an accurate and repeatable indication of the dose amount when the indicia 146 of the plunger 140 are aligned within the window 176. In yet other embodiments, the dose gauge 170 is snapped into place over a portion of the syringe body 110 (i.e., via an interference fit).

The cap 160 is coupled to the proximal end portion 112 of the syringe body 110, and defines an opening through which the internal volume 118 of the syringe body 110 can be accessed. Moreover, as shown in FIG. 2, the cap 160 includes an engagement portion 164, which can be any suitable structure or mechanism to engage and/or interface with a corresponding engagement portion 148 of the plunger 140 to limit rotation of the plunger 140 within the syringe body 110 about the longitudinal axis LA. For example, as shown, the engagement portion 164 can include a protrusion configured to be disposed within a corresponding groove 148 of the plunger 140. In other embodiments, the engagement portion 164 can include a recessed portion configured to receive a protrusion of the plunger 140. In yet other embodiments, the engagement portion 164 can include a splined surface that interfaces with a corresponding splined surface of the plunger 140 to limit, reduce and/or prevent rotation of the plunger 140 within the syringe body 110.

The cap 160 can be coupled to the syringe body 110 in any suitable manner. In some embodiments, the cap 160 can be bonded to the side wall 115 of the syringe body 110 (e.g., via an adhesive, a weld joint or the like). In other embodiments, the cap 160 can be coupled to the dose gauge 170 or a flange (not shown) of the syringe body 110 to couple the cap 160 to the side wall 115. In yet other embodiments, the cap 160 is snapped into place over the proximal end portion 112 of the syringe body 110 (i.e., via an interference fit).

The plunger 140 has a proximal end portion 142 and a distal end portion 144. The distal end portion 144 of the plunger 140 is configured to move within the interior volume 118 of the syringe body 110 to convey a medicament. More particularly, as described in more detail below, the distal end portion 144 can reciprocate within the syringe body 110 along the longitudinal axis L_{A} to convey the medicament into the volume 118 (as shown in FIG. 3), and convey the medicament out of the volume 118 (as shown by the arrow CC in FIG. 4). In some embodiments, the distal end portion 144 includes an elastomeric member defining a fluid-tight (or substantially fluid-tight) seal with the side wall 115 of the syringe body 110.

The plunger 140 includes an engagement portion 148 configured to interface with the engagement portion 164 of the syringe body 110 to limit rotation of the plunger 140 about its longitudinal axis A_{L}. The engagement portion 148 can be any suitable structure or mechanism to engage and/or interface with the engagement portion 164 of the syringe body 110 to limit rotation of the plunger 140 within the syringe body 110 about the longitudinal axis A_{L}. For example, as shown, the engagement portion 148 can be a recessed portion configured to receive the protrusion 164 of the cap 160. In other embodiments, the engagement portion 148 can include a protrusion configured to be disposed within a corresponding groove of the cap 160 or the syringe body 110. In yet other embodiments, the engagement portion 148 can include a splined surface that interfaces with a corresponding splined surface of the cap 160 to limit, reduce and/or prevent rotation of the plunger 140 within the syringe body 110.

The plunger 140 includes an outer surface 145 having a series of indicia 146. As shown in FIG. 3, at least one of the indicium from the series of indicia 146 is visible through the window 176 of the syringe body 110 when the plunger 140 is in the first position. In this manner, the indicia 146 can provide a visual indication of the dosage of medicament drawn into the syringe body 110. More specifically, because the indicia 146 are in a fixed position on the plunger 140 and the window 176 of the dose gauge 170 is in a fixed position on the syringe body 110, the position of the plunger 140 within the syringe body 110 at which one of the indicium 146 is visible via the window 176 corresponds to a volume (i.e., dosage volume) within the syringe body 110. In some embodiments, the indicia 146 can be non-volumetric indicia that correspond to a characteristic of the patient. For example, in some embodiments, the indicia 146 can correspond to a weight, height, age, target body weight, and/or body mass index (BMI) of the patient. In other embodiments, the indicia 146 can correspond to a test result associated with the patient, including, for example, a range of blood sugar (e.g., for insulin dosage) or any other suitable test result. In this manner, a user can withdraw a dosage of medicament without the need for calculation or conversion to determine the volumetric amount.

In some embodiments, the series of indicia 146 can include both a series of dosage amounts (e.g., a series of weight ranges corresponding the desired dosage) and one or more instructions. For example, in some embodiments, one indicia from the series of indicia 146 can be configured and/or position to be visible via (or appear within) the window 176 when the syringe assembly 100 is in a “ready” (or empty) state (i.e., with the plunger 140 in the second position). In this manner, an indicium can be visible via the window 176 when the plunger 140 is in the distal-most position within the syringe body 110. In some embodiments, an instruction indicia 146 can include an identification of the drug with which the syringe assembly 100 should be used. In this manner, the instruction indicia 146 can minimize the likelihood that the syringe assembly 100 will be used with an improper medicament.

The series of indicia 146 can be arranged along the outer surface 145 in any suitable manner or orientation. For example, in some embodiments, the series of indicia 146 is arranged coaxially along the longitudinal axis A_{L}. In this manner, when the plunger 140 is reciprocated along the longitudinal axis A_{L} within the internal volume 118 of the syringe body 110, each of the indicia 146 will be visible through the window 176 of the dose gauge 170 at a given plunger position.

In use, the distal end portion 144 of the plunger 140 can be reciprocated within the syringe body 110 along the longitudinal axis A_{L} to convey a medicament into and/or out of the internal volume 118 of the syringe body 110. In the initial configuration, the plunger 140 is positioned at its distal-most position within the syringe body 110 (i.e., in the second position). To prepare a dose of the medicament for delivery, the delivery tip 132 is placed in fluid communication with a source of medicament (e.g., a medicament container, not shown in FIGS. 1-4), and the plunger 140 is moved proximally, as shown by the arrow AA in FIG. 3. The movement of the distal end portion 144 of the plunger 140 within the syringe body 110 increases the internal volume 118, which, in turn, produces a vacuum that draws the medicament 105 into the syringe body 110. As shown in FIG. 3, the plunger 140 is moved proximally until an indicium 146 (the indicia “XX”) is visible through the window 176 of the dose gauge 170. In this manner, the syringe assembly 100 is placed in its second (or “dosage set”) configuration (and the plunger 140 is in a first position within the syringe body 110). The indicium 146 (indicated as “XX”) can be, for example, a dose indicia. In some embodiments, the second indicium 146 can be a non-volumetric indicium (e.g., a weight range, a height range and/or an age range of the patient). In this manner, the user can easily set the desired dosage by withdrawing the plunger 140 until the indicium 146 (indicated as “XX”) is visible within the window 176 of the dose gauge 170. This arrangement allows the dosage to be set without the need for calculation, conversion or consideration of the patient's characteristics to a volumetric measurement.

As described above, during the movement of the plunger 140, the engagement portion 148 of the plunger 140 interfaces with the engagement portion 164 of the cap 160 to limit rotation of the plunger 140 about the longitudinal axis A_{L} of the plunger. In this manner, the indicia 146 remain radially aligned with the window 176 of the dose gauge 170, thus ensuring that the indicia 146 will be visible through the window 176 of the dose gauge 170 when the plunger 140 is in the corresponding longitudinal position within the syringe body 110.

To deliver the dosage withdrawn, the user then places the delivery tip 132 in the desired location (e.g., in the patient's mouth) and moves the plunger 140 distally, as shown by the arrow BB in FIG. 4. The movement of the distal end portion 144 of the plunger 140 within the syringe body 110 decreases the internal volume 118, which, in turn, produces a pressure that conveys the medicament out of the syringe body 110 as shown by the arrow CC in FIG. 4. This movement places the syringe assembly 100 in its third (or dose delivered) configuration.

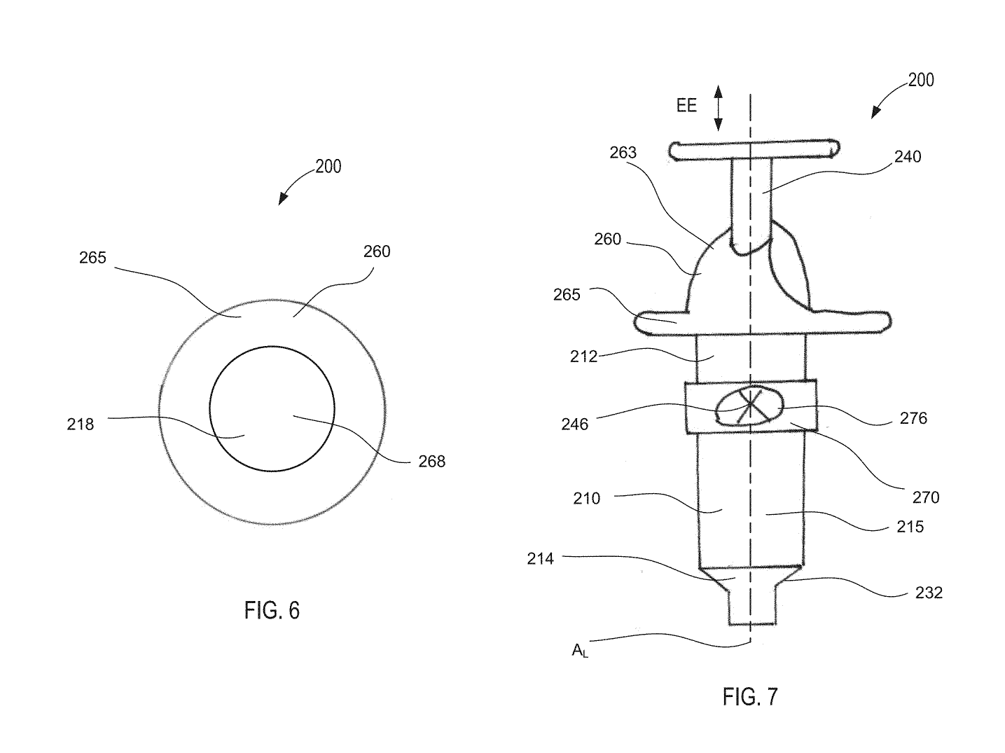

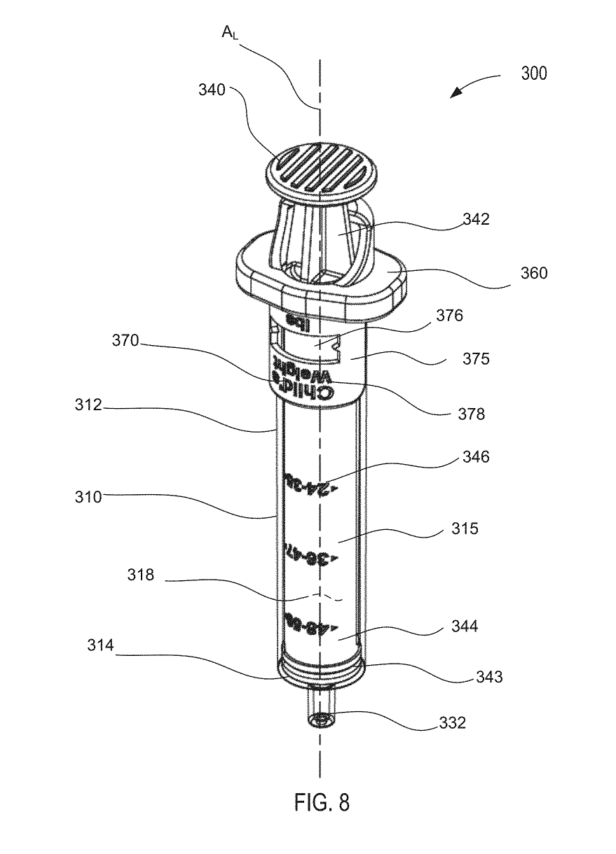

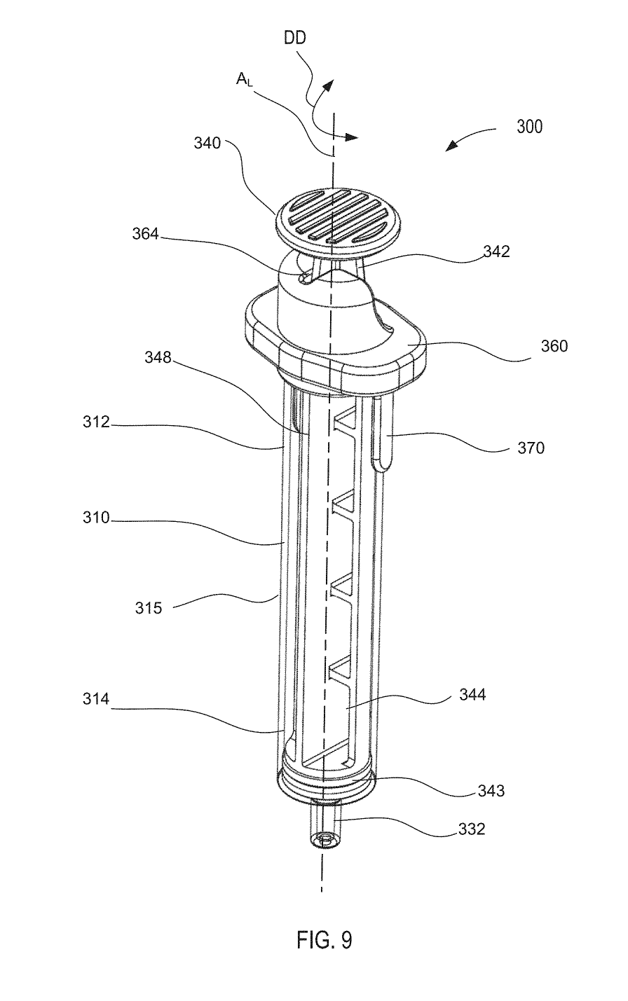

In some embodiments, the syringe assembly 100 can be cleaned for reuse by repeatedly reciprocating the plunger 140 within the syringe body 110 to rinse with water, saline solution or the like. In other embodiments, the syringe assembly 100 can be disassembled for cleaning (e.g., by decoupling the engagement portion 148 of the plunger 140 from the engagement portion 164 of the cap 160), in any suitable manner, as described herein. In other embodiments, a plunger of syringe assembly need not be engaged with a cap or syringe body throughout the range of motion of the plunger. In some embodiments, for example, a cap can include multiple openings through which the plunger is moved to be placed into and/or removed from within the syringe body. For example, FIGS. 5-7 are schematic illustrations of a syringe assembly 200, according to an embodiment that includes a syringe body 210 and a plunger 240. FIG. 5 shows an exploded view of the syringe assembly 200 to illustrate the syringe body 210 and the plunger 240. FIG. 6 shows a top view of a cap 260 and the syringe body 210 taken along the line X-X in FIG. 2. FIG. 7 shows the syringe assembly 200 in a first (or dosage set) configuration. The syringe body 210 includes a side wall 215, and defines an interior volume 218 within which a medicament can be contained. The syringe body 210 includes a proximal end portion 212, a distal end portion 214, and defines a longitudinal axis A_{L}. When the plunger 240 is disposed within the syringe body 210 (FIG. 7), the longitudinal axis A_{L} of the syringe body 210 is coaxial with the longitudinal axis A_{L} of the plunger 240. Thus, the longitudinal axis A_{L} shown and described can be considered the longitudinal axis for the syringe body 210, the plunger 240, or both the syringe body 210 and the plunger 240.

The distal end portion 214 of the syringe body 210 has a delivery tip 232. The delivery tip 232 can be any suitable tip or member through which the medicament can be conveyed either into or out of the internal volume 218 of the syringe body 210. In some embodiments, the delivery tip 232 can be a protrusion extending from the syringe body 210 that can be received within a medicament container (e.g., bottle, vial or the like) and that can also deliver the medicament orally to the patient. In other embodiments, the delivery tip 232 can a tapered fitting (such as a Luer fitting) that is adapted to couple the distal end portion 214 of the syringe body 210 to a needle (not shown).

The proximal end portion 212 of the syringe body 210 includes a cap 260 and a dose gauge 270, and defines an opening (also referred to as a first opening) 268. As shown in FIG. 6, the volume 218 of the syringe body 210 can be accessed through the opening 268. Moreover, the opening is aligned with the constant cross-sectional shape of the volume 218. Similarly stated, the opening 268 defines a central axis (i.e., and axis that is normal to a plane defined by the opening) that is coaxial with the longitudinal axis A_{L} of the syringe body 210. Thus, referring to FIG. 5, the longitudinal axis A_{L} is normal to and is the central axis of the opening 268.

The cap 260 includes a distal surface 265 and a proximal surface 263. The distal surface 265 can be, for example, a flange or shoulder that accommodates the user's fingers to actuate the syringe assembly 200. In some embodiments, the cap 260 can be a separately constructed component (i.e., from the syringe body 210) that is coupled to the proximal end portion 212 of the syringe body 210 via a portion of the distal surface 265. For example, in some embodiments, a portion of the distal surface 265 can be adhesively coupled to the syringe body 210. In other embodiments, however, the cap 260 can be monolithically constructed with the syringe body 210. In some embodiments, the distal surface 265 defines an opening (not shown) that is aligned with the opening 268 of the syringe body 210.

The proximal surface 263 of the cap 260 extends above the opening 268 of the syringe body 210. In this manner, the cap 260 provides a covering over at least a portion of the opening 268. The proximal surface 263 can also provide a surface against which a proximal end portion 242 of the plunger 240 can contact during use. The cap 260 defines a lateral opening (also referred to as a second opening) 267 between the distal surface 265 and the proximal surface 263. As described in more detail herein, the lateral opening 267 provides a passageway through which the distal end portion 244 of the plunger 240 can be moved when the distal end portion 244 is disposed into the volume 218 of the syringe body. As shown in FIG. 5, the lateral opening 267 defines a central axis A_{LAT} that is normal to a plane defined by the lateral opening 267. The central axis A_{LAT} is nonparallel to the central axis of the first opening 268.

The dose gauge 270 is coupled about at least a portion of the side wall 215 of the syringe body 210, and defines a window 276. In particular, the dose gauge 270 is coupled about a transparent portion of the side wall 215 so that portions of the plunger 240 (i.e., the indicia 246) within the syringe body 210 can be seen through the window 276. In this manner, as described in more detail below, the dose gauge 270 and the plunger 240 can provide a visual indication of the dosage of medicament drawn into the syringe body 210. In some embodiments, a portion of the dose gauge 270 surrounding the window 276 is opaque, or otherwise includes a visible frame (not shown) that surrounds the window 276. In this manner, during use, the indicia 246 displayed within the window 276 are clearly accentuated to the user. Moreover, in some embodiments, an outer surface of the dose gauge 270 can include instructions or indicia (not shown) associated with the medicament, dose, and/or treatment regimen. Such instructions or indicia can include, for example, the characteristic of the patient to be considered when setting the dosage (e.g., “child's weight”), alignment marks or arrows, identification of the regimen (e.g., “day 2”), and/or the drug name.

The window 276 can be a portion of the dose gauge that is devoid of material. In other embodiments, however, the window 276 can include a transparent material through which a user can view the indicia 246. Moreover, the window 276 can be of any suitable size and/or shape. Thus, although shown as being elliptical, in other embodiments, the window 276 can be rectangular. Moreover, although the window 276 is shown as being sized such that only one indicium 246 from the series of indicia is visible through the window at a time, in other embodiments, the dose gauge 270 (or any of the dose gauges described herein) can include a window through which multiple indicia can be viewed at a time. In yet other embodiments, the dose gauge 270 (or any of the dose gauges described herein) can include more than one window.

Although the dose gauge 270 is shown as extending only a portion of the length of the syringe body 210, in other embodiments, the dose gauge 270 (or any of the dose gauges described herein) can have a length that is substantially the same as the syringe body 210 (i.e., can extend substantially the full length of the syringe body 210). Moreover, the dose gauge 270 (or any of the dose gauges described herein) can extend around any portion of the circumference of the syringe body 210. For example, in some embodiments, the dose gauge 270 can extend around the full circumference of the syringe body 210. In this manner, the window 276 can be disposed about one portion of the syringe body 210 and other portions (e.g., opaque portions) of the dose gauge 270 can be disposed about another portion of the syringe body. By covering certain portions of the syringe body 210, the user can more easily identify the visual dose indication. In other embodiments, however, the dose gauge 270 can extend around only a portion of the circumference of the syringe body 210. For example, in some embodiments, the dose gauge 270 can extend around about three quarters of the circumference (about 270 degrees). In other embodiments, the dose gauge 270 can extend around about one half to three quarters of the circumference (from about 280 degrees to about 270 degrees). In other embodiments, the dose gauge 270 can extend around about one quarter to one half of the circumference (from about 90 degrees to about 280 degrees). In this manner, the window 276 can be disposed about one portion of the syringe body 210 and other portions of the syringe body 210 can be exposed. By exposing certain portion (e.g., the “back side) of the syringe body 210, the user can visually inspect the medicament in the syringe body 210 for air bubbles, the correct color, and the like.

The dose gauge 270 can be coupled to the syringe body 210 in any suitable manner. In some embodiments, the dose gauge 270 can be bonded to the side wall 215 of the syringe body 210 (e.g., via an adhesive, a weld joint or the like). In other embodiments, the dose gauge 270 can be coupled to the cap 260 or a flange (not shown) of the syringe body 210 to couple the dose gauge 270 about the side wall 215. As discussed below, by indexing the dose gauge 270 to the cap 260, the dose gauge 270 can be maintained in a fixed location along the longitudinal axis A_{L} of the syringe body 210, thereby producing an accurate and repeatable indication of the dose amount when the indicia 246 of the plunger 240 are aligned within the window 276. In yet other embodiments, the dose gauge 270 is snapped into place over a portion of the syringe body 210 (i.e., via an interference fit). In still other embodiments, the dose gauge 270 and the cap 260 can be monolithically constructed, and can be coupled to the syringe body 210 in one operation. In other embodiments, the dose gauge 270 and the syringe body 210 can be monolithically constructed. In other embodiments, the dose gauge 210, the cap, and the syringe body 210 can be monolithically constructed.

In some embodiments, the syringe body 210 (and any of the syringe bodies described herein) can be constructed from a clear material (e.g., plastic or glass), and can include markings or graduated lines on the outer surface of the side wall 215. In some embodiments, the syringe body 210 (and any of the syringe bodies described herein) can include an opaque label (not shown) that defines one or more transparent windows through which a user can view the medicament or any other contents (e.g., the plunger 240) within the internal volume 218. In other embodiments, the syringe body 210 (and any of the syringe bodies described herein) can include a monolithically constructed opaque or semi-opaque portion. Such portions can include a light blocking color, or can be devoid of color, but can otherwise by hazy, blurred or textured to prevent medicament or other contents within the internal volume 218 from being clearly seen.

The plunger 240 has a proximal end portion 242 and a distal end portion 244. The distal end portion 244 of the plunger 240 is configured to move within the interior volume 218 of the syringe body 210 to convey a medicament. More particularly, as described in more detail below, the distal end portion 244 can reciprocate within the syringe body 210 along the longitudinal axis L_{A} to convey the medicament into and out of the volume 218. In some embodiments, the distal end portion 244 includes an elastomeric member defining a fluid-tight (or substantially fluid-tight) seal with the side wall 215 of the syringe body 210.

The plunger 240 includes an outer surface 245 having a series of indicia 246. As shown in FIG. 7, at least one of the indicium from the series of indicia 246 is visible through the window 276 of the syringe body 210 when the plunger 240 is in the first (i.e., dose ready) position. In this manner, the indicia 246 can provide a visual indication of the dosage of medicament drawn into the syringe body 210. More specifically, because the indicia 246 are in a fixed position on the plunger 240 and the window 276 of the dose gauge 270 is in a fixed position on the syringe body 210, the position of the plunger 240 within the syringe body 210 at which one of the indicium 246 is visible via the window 276 corresponds to a volume (i.e., dosage volume) within the syringe body 210. In some embodiments, the indicia 246 can be non-volumetric indicia that correspond to a characteristic of the patient. For example, in some embodiments, the indicia 246 can correspond to a weight, height, age, target body weight, and/or body mass index (BMI) of the patient. In other embodiments, the indicia 246 can correspond to a test result associated with the patient, including, for example, a range of blood sugar (e.g., for insulin dosage) or any other suitable test result. In this manner, a user can withdraw a dosage of medicament without the need for calculation or conversion to determine the volumetric amount.

The series of indicia 246 can be arranged along the outer surface 245 in any suitable manner or orientation. For example, in some embodiments, the series of indicia 246 is arranged coaxially along the longitudinal axis A_{L}. In this manner, when the plunger 240 is reciprocated along the longitudinal axis A_{L} within the internal volume 218 of the syringe body 210, each of the indicia 246 will be visible through the window 276 of the dose gauge 270 at a given plunger position.

In use, the plunger 240 can be removed from the syringe body 210 for cleaning. To prepare the syringe assembly 200 for use, the distal end portion 244 of the plunger 240 is placed within the volume 218 of the syringe body 210. Specifically, the distal end portion 244 is first moved through the lateral opening 267, as shown by the arrow DD in FIG. 5. The direction of motion of the distal end portion 244 is nonparallel to the longitudinal axis A_{L}. After the distal end portion 244 is through the lateral opening 267, it is then moved distally through the first opening 268 and into the volume 218. The direction of motion of the distal end portion 244 through the first opening 268 is parallel to the longitudinal axis A_{L}. Thus, the movement of the distal end portion 244 occurs in two discrete motions. The distal end portion 244 of the plunger 240 can then be reciprocated within the syringe body 210 along the longitudinal axis A_{L}, as shown by the arrow EE, to convey a medicament into and/or out of the internal volume 218 of the syringe body 210, in a similar manner as described above for the syringe assembly 100. After delivery of the medicament, the syringe assembly 200 can be disassembled for cleaning by removing the distal end portion 244 of the plunger 240 from the internal volume 218, a manner opposite to the steps described above.

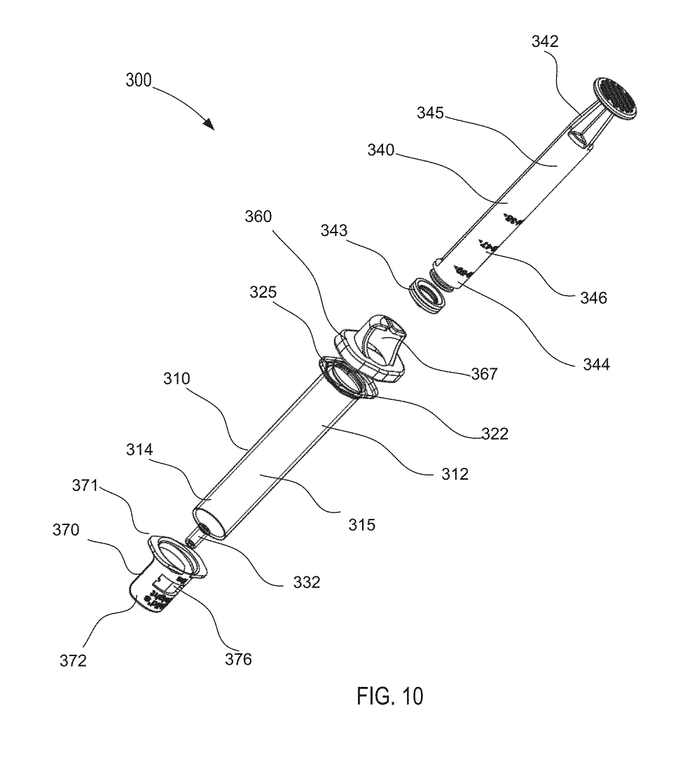

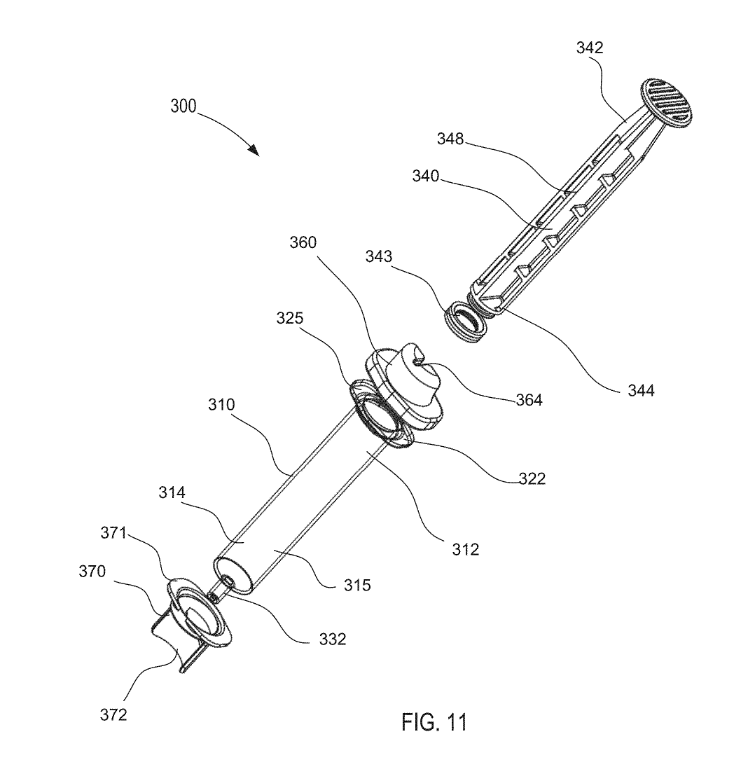

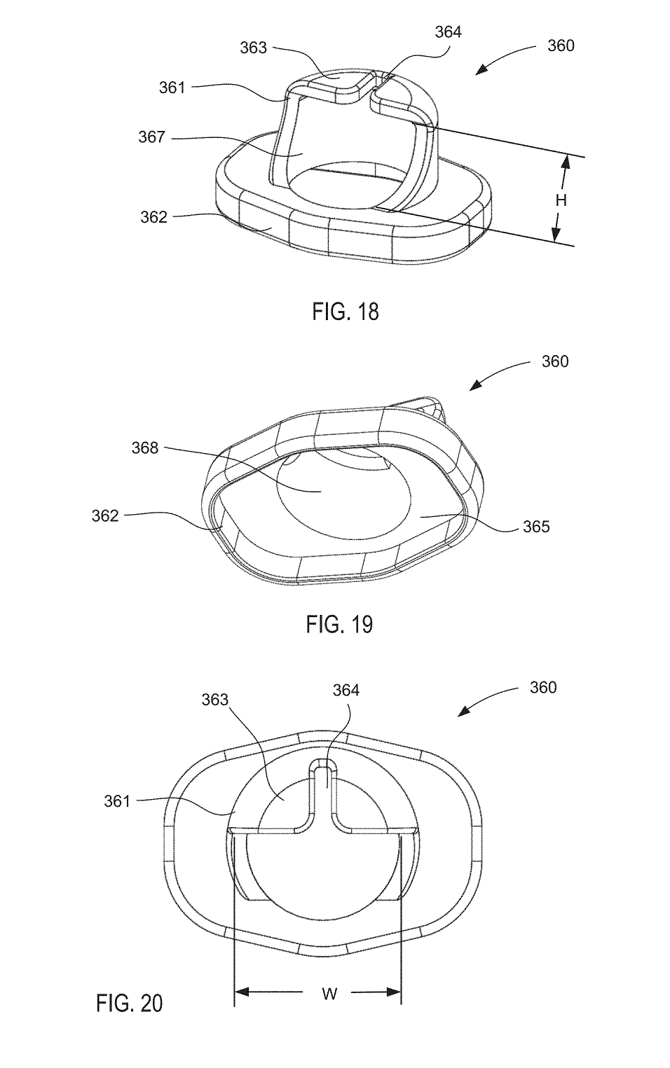

FIGS. 8-21 illustrate a syringe assembly 300, according to an embodiment. FIGS. 8 and 9 are a front perspective view and a back perspective view, respectively, of the syringe assembly 300 with the plunger 340 in a second position (fully bottomed) within the syringe body 310. The syringe assembly 300 includes a syringe body 310, a dose gauge 370, a cap 360, and a plunger 340. The syringe body 310, which is shown a transparent view in the drawings to allow viewing of the contents therein, includes a side wall 315, and defines an interior volume 318 within which a medicament can be contained. The syringe body 310 includes a proximal end portion 312, a distal end portion 314, and defines a longitudinal axis A_{L}. When the plunger 340 is disposed within the syringe body 310, the longitudinal axis A_{L} of the syringe body 310 is coaxial with the longitudinal axis A_{L} of the plunger 340. Thus, the longitudinal axis A_{L} shown and described can be considered the longitudinal axis for the syringe body 310, the plunger 340, or both the syringe body 310 and the plunger 340.

The proximal end portion 312 of the syringe body includes a flange 322 and defines an opening 323. The volume 318 of the syringe body 310 can be accessed through the opening 323. Moreover, the opening 323 is aligned with the constant cross-sectional shape of the volume 318. Similarly stated, the opening 323 defines a central axis (i.e., and axis that is normal to a plane defined by the opening) that is coaxial with the longitudinal axis A_{L} of the syringe body 310. Thus, the longitudinal axis A_{L} is normal to and is the central axis of the opening 323.

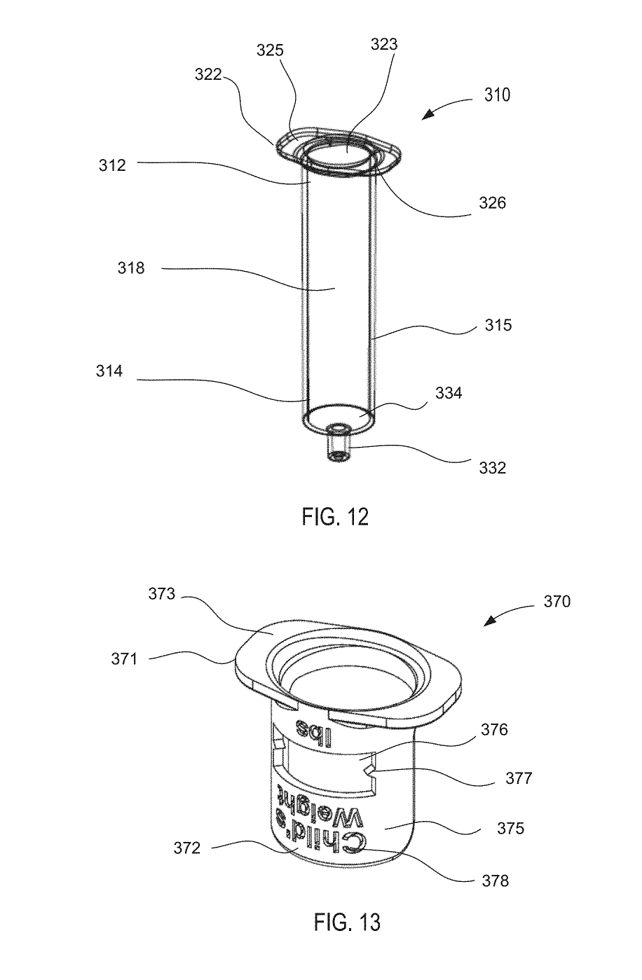

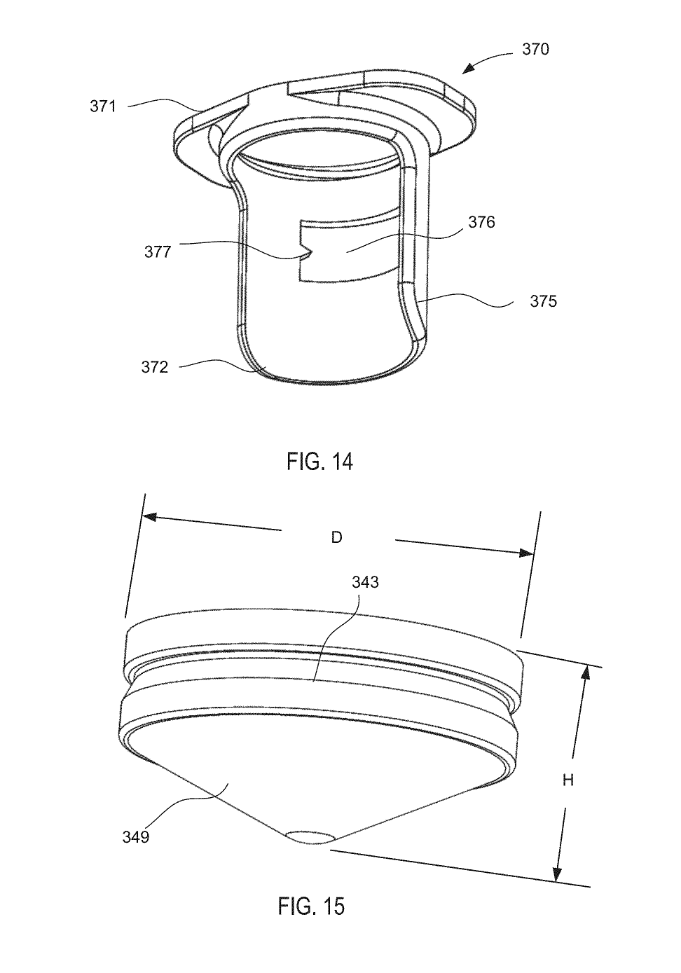

The flange 322 can be manipulated by a user to actuate the syringe assembly 300. The flange is also configured to engage and/or be coupled to the dose gauge 370 and/or the cap 360. Specifically, the flange includes a proximal surface 325 and a distal surface 326 (see FIG. 12). The proximal surface 325 is configured to engage a coupling surface 365 of the cap (see FIG. 19) when the cap 360 is coupled to the flange 322. In this manner, the flange 322 and/or the proximal surface 325 function as a locating index to which the cap 360 is mounted. The distal surface 326 is configured to engage a top surface 373 of the dose gauge 370 (see FIG. 13) when the dose gauge 370 is coupled to the flange 322. In this manner, the flange 322 and/or the distal surface 326 function as a locating index to which the dose gauge 370 is mounted. By indexing the dose gauge 370 to the flange 322 and/or the syringe body 310, the dose gauge 370 can be maintained in a fixed location along the longitudinal axis A_{L} of the syringe body 310, thereby producing an accurate and repeatable indication of the dose amount when the indicia 346 of the plunger 340 are aligned within the window 376.

As shown in FIG. 12, he distal end portion 314 of the syringe body 310 has a delivery tip 332 and an internal tapered surface 334. The tapered surface 334 is a frusto-conical shaped portion that corresponds to the shape of the distal end surface 349 of the elastomeric member 343. Thus, when the plunger 340 is in the second (or fully distal) position (see FIGS. 8 and 9), the distal end surface 349 is in contact with the tapered surface 334 of the syringe body 310. This arrangement limits trapped pockets of medicament within the volume 318 and ensures that the full dose is delivered via the delivery tip 332. The delivery tip 332 can be any suitable tip or member through which the medicament can be conveyed either into or out of the internal volume 318 of the syringe body 310. In some embodiments, the delivery tip 332 can be a protrusion extending from the syringe body 310 that can be received within a medicament container (e.g., bottle, vial or the like) and that can also deliver the medicament orally to the patient. In other embodiments, the delivery tip 332 can a tapered fitting (such as a Luer fitting) that is adapted to couple the distal end portion 314 of the syringe body 310 to a needle (not shown).

In some embodiments, the syringe body 310 (and any of the syringe bodies described herein) can be constructed from a clear material (e.g., plastic or glass), and can include markings or graduated lines on the outer surface of the side wall 315. In some embodiments, the syringe body 310 (and any of the syringe bodies described herein) can include an opaque label (not shown) that defines one or more transparent windows through which a user can view the medicament or any other contents (e.g., the plunger 340) within the internal volume 318. In other embodiments, the syringe body 310 (and any of the syringe bodies described herein) can include a monolithically constructed opaque or semi-opaque portion. Such portions can include a light blocking color, or can be devoid of color, but can otherwise by hazy, blurred or textured to prevent medicament or other contents within the internal volume 318 from being clearly seen.

In some embodiments, the syringe body 310 (and any of the syringe bodies described herein) can be of any suitable size. For example, in some embodiments, the internal volume 318 (or the internal volume of any of the syringe bodies described herein) can be 1 mL, 3 mL, 5 mL, 10 mL, and 20 mL. In other embodiments, the internal volume 318 (or the internal volume of any of the syringe bodies described herein) can be greater than 20 mL. In some embodiments, the syringe body 310 (and any of the syringe bodies described herein) can be the syringe body from an off-the-shelf oral medication syringe produced by Covidien (now known as Medtronic Minimally Invasive Therapies), for example, the syringes known as the “Monojet Oral Medication Syringe,” or the “10 mL Slip DbDjj Tip Oral Medication Syringe.” In other embodiments, the syringe body 310 (and any of the syringe bodies described herein) can be the syringe body from an off-the-shelf oral medication syringe produced by Becton, Dickinson and Company, for example, the syringes identified as the “BD Enteral Syringe with BD UniVia™ Connector” or the “Oral Syringe with Tip Cap” (BD catalog numbers 305217, 305218, 305219, 305220, 305207, 305208, 305209, 305210. In other embodiments, the syringe body 310 (and any of the syringe bodies described herein) can be the syringe body from an off-the-shelf oral medication syringe produced by Ezy-Dose (Apothecary Products LLC), for example, the syringes identified the “Dosage Korc™.” In yet other embodiments, the syringe body 310 (and any of the syringe bodies described herein) can be the syringe body from an off-the-shelf oral medication syringe produced by Duda Energy, for example, the needled syringes identified the “15G×1½” Blunt Tip Fill Needle and Plastic Cover.”

The dose gauge 370 includes a proximal end portion 371 and a distal end portion 372, and is coupled about at least a portion of the side wall 315 of the syringe body 310. As shown, the dose gauge 370 defines a window 376 through which portions of the plunger 340 (i.e., the indicia 346) within the syringe body 310 can be seen (i.e., via a transparent portion of the side wall 315 of the syringe body 310). In this manner, as described in more detail below, the dose gauge 370 and the plunger 340 can provide a visual indication of the dosage of medicament drawn into the syringe body 310.

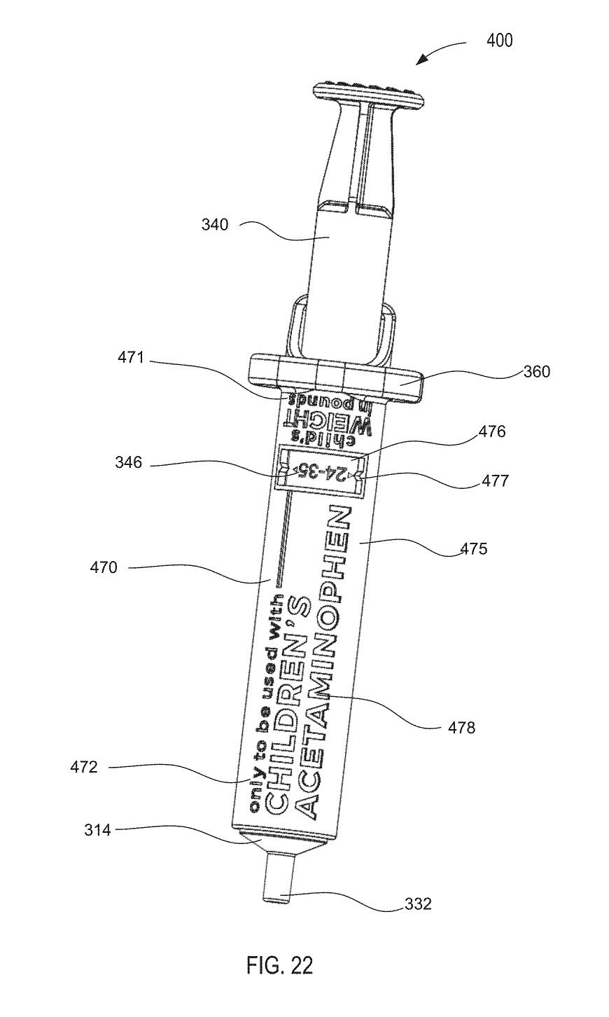

The dose gauge 370 includes an opaque portion 375 surrounding the window 376. In this manner, the opaque portion 375 can provide a frame that surrounds the window 376. Thus, during use, the indicia 346 displayed within the window 376 are clearly accentuated to the user. Moreover, the opaque portion 375 surrounding the window 376 includes at least one alignment protrusion 377. As shown in FIG. 21, the alignment protrusion 377 is configured to be aligned with the one indicium 346 from the series of indicia when the plunger 340 is in the first (dose ready) position. The outer surface of the dose gauge 370 can include instructions or indicia 378 associated with the medicament, dose, and/or treatment regimen. Such instructions or indicia can include, for example, the characteristic of the patient to be considered when setting the dosage (e.g., “child's weight”), the units of measure (e.g., pounds), alignment marks or arrows, identification of the regimen (e.g., “day 2”), and/or the drug name.

The window 376 can be a portion of the dose gauge that is devoid of material. In other embodiments, however, the window 376 can include a transparent material through which a user can view the indicia 346. Moreover, the window 376 can be of any suitable size and/or shape. Thus, although shown as being rectangular, in other embodiments, the window 376 can be circular, oval, or any other polygon. Although the window 376 is shown as being sized such that only one indicium 346 from the series of indicia is visible through the window at a time, in other embodiments, the dose gauge 370 (or any of the dose gauges described herein) can include a window through which multiple indicia can be viewed at a time. In yet other embodiments, the dose gauge 370 (or any of the dose gauges described herein) can include more than one window.

The proximal end portion 371 of the dose gauge 370 includes a flange that has a top surface 373. As described above, the top surface 373 is configured to engage the distal surface 326 of the syringe flange 322 to couple the dose gauge 370 to the flange 322. In this manner, the dose gauge 370 is indexed to the flange 322 and/or the distal surface 326. By indexing the dose gauge 370 to the flange 322 and/or the syringe body 310, the dose gauge 370 can be maintained in a fixed location along the longitudinal axis A_{L} of the syringe body 310, thereby producing an accurate and repeatable indication of the dose amount when the indicia 346 of the plunger 340 are aligned within the window 376.

The dose gauge 370 can be coupled to the syringe body 310 in any suitable manner. In some embodiments, the top surface 373 can be bonded to the flange 322 of the syringe body 310 (e.g., via an adhesive, a weld joint or the like). In other embodiments, the dose gauge 370 can be coupled to the cap 360 in a manner that surrounds the flange 322 of the syringe body 310 to couple the dose gauge 370 to the syringe body. In yet other embodiments, the dose gauge 370 is snapped into place over a portion of the syringe body 310 and/or the cap 360 (i.e., via an interference fit).

Referring to FIG. 14, the opaque portion 375 extends around approximately one half of the circumference of the syringe body 310 (about 270 degrees). By exposing certain portion (e.g., the “back side) of the syringe body 310, the user can visually inspect the medicament in the syringe body 310 for air bubbles, the correct color of the medicament, and the like. In other embodiments, the dose gauge 370 (or any of the dose gauges described herein) can extend around any suitable portion of the circumference of the syringe body 310. For example, in some embodiments, the dose gauge 370 can extend around the full circumference of the syringe body 310. In this manner, the window 376 can be disposed about one portion of the syringe body 310 and other portions (e.g., opaque portions) of the dose gauge 370 can be disposed about another portion of the syringe body. By covering certain portions of the syringe body 310, the user can more easily identify the visual dose indication. In other embodiments, however, the dose gauge 370 (or any of the dose gauges described herein) can extend around only a portion of the circumference of the syringe body 310. For example, in some embodiments, the dose gauge 370 can extend around about three quarters of the circumference (about 270 degrees). In other embodiments, the dose gauge 370 can extend around about one half to three quarters of the circumference (from about 380 degrees to about 270 degrees). In other embodiments, the dose gauge 370 can extend around about one quarter to one half of the circumference (from about 90 degrees to about 180 degrees). In this manner, the window 376 can be disposed about one portion of the syringe body 310 and other portions of the syringe body 310 can be exposed.

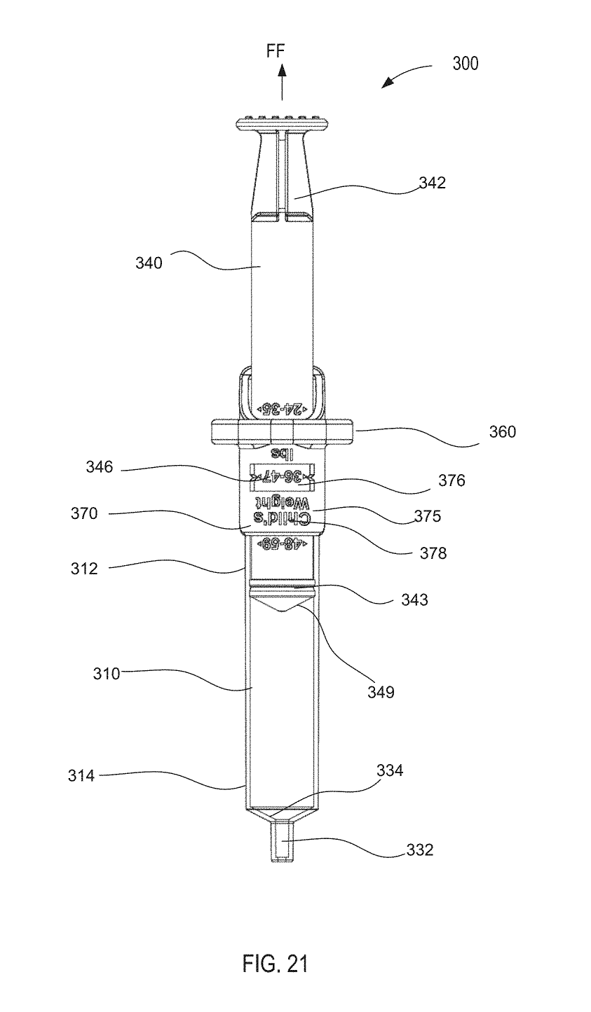

Referring to FIGS. 18-20, the cap 360 includes a proximal end portion 361 and a distal end portion 362. The distal end portion 362 includes a bottom surface 365 that defines a central opening 368 that is aligned with the opening 323 of the syringe body 310. The distal end portion 362 also includes a lip that surrounds the bottom surface 365. As described above, the bottom surface 365 of the cap 360 is configured to engage the proximal surface 325 of the flange 322 to couple the cap 360 to the syringe body 310. Moreover, the lip can have a shape that corresponds to a shape of the flange 322, and can thus surround the edges of the flange 322 when the cap 360 is coupled to the syringe body 310. The cap 360 can be coupled to the syringe body 310 in any suitable manner. For example, in some embodiments, a portion of the distal surface 365 can be adhesively coupled to the proximal surface 325 of the flange 322. In other embodiments, however, the cap 360 can be monolithically constructed with the syringe body 310.

The proximal end portion 361 includes a top (or proximal) surface 363 that defines an engagement slot 364. The top surface 363 extends above the bottom surface 365 and the opening 368. In this manner, the cap 360 provides a covering over at least a portion of the opening 368 and the opening 323 of the syringe body 310. The engagement slot 364 is configured to engage and/or interface with a corresponding engagement protrusion 348 of the plunger 340 to limit rotation of the plunger 340 within the syringe body 310 about the longitudinal axis L_{A}. Although shown as being an engagement slot 364, in other embodiments, the cap 360 (and any of the caps described herein) can include an engagement protrusion configured to be disposed within a corresponding groove of the plunger, or any other suitable mechanism to limit and/or prevent rotation of the plunger within the syringe body 310 about the longitudinal axis L_{A}. In yet other embodiments, cap 360 can include a splined surface that interfaces with a corresponding splined surface of the plunger 340 to limit, reduce and/or prevent rotation of the plunger 340 within the syringe body 310.

The cap 360 defines a lateral opening (also referred to as a second opening) 367 between the distal surface 365 and the proximal surface 363. As described in more detail herein, the lateral opening 367 provides a passageway through which the distal end portion 344 of the plunger 340 (including the elastomeric member 343) can be moved when the distal end portion 344 is disposed into or removed from the volume 318 of the syringe body. The lateral opening 367 defines a central axis (not shown) that is normal to a plane defined by the lateral opening 367. The central axis is nonparallel to the central axis of the first opening 368 and/or the opening 323 of the syringe body 310. Thus, in a similar manner as described above for the cap 260, the distal end 344 of the plunger 340 is moved in two distinct directions (along the central axis of the opening and along the longitudinal axis A_{L}) when being moved into or out of the syringe body 310.

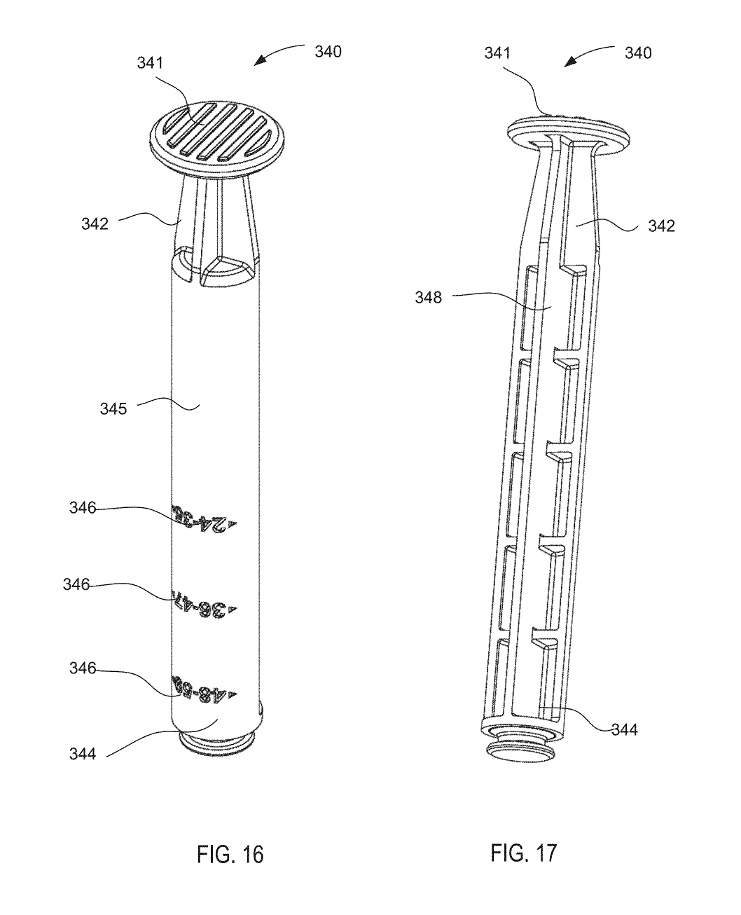

Moreover, the lateral opening 367 is sized such that the distal end portion 344 of the plunger 340 can be removed from the volume of the syringe body 310 via the lateral opening 367. In particular, the lateral opening 367 has a height H (see FIG. 18) that is greater than a height H of the elastomeric member 343 (see FIG. 15). The lateral opening 367 has a width W (see FIG. 20) that is greater than a diameter D of the elastomeric member 343 (see FIG. 15).

The plunger 340 has a proximal end portion 342 and a distal end portion 344. The distal end portion 344 of the plunger 340 is configured to move within the interior volume 318 of the syringe body 310 to convey a medicament. As shown, the distal end portion 344 includes an elastomeric member 343 that defines a substantially fluid-tight seal with the side wall 315 of the syringe body 310. Thus, the distal end portion 344 can reciprocate within the syringe body 310 along the longitudinal axis LA to convey the medicament into and out of the volume 318. Moreover, as described in more detail below, the distal end portion 344 (including the elastomeric member 343) can be removed from the syringe body 310 via the lateral opening 367 for cleaning, storage, or the like. The proximal end portion 342 of the plunger 340 includes an activation handle 341 that can be grasped and/or manipulated by a user to move the plunger 340 within the syringe body 310.

The plunger 340 includes an outer surface 345 having a series of indicia 346. As shown in FIG. 21, at least one of the indicium from the series of indicia 346 is visible through the window 376 of the syringe body 310 when the plunger 340 is in the first (i.e., dose ready) position. In this manner, the indicia 346 can provide a visual indication of the dosage of medicament drawn into the syringe body 310. More specifically, because the indicia 346 are in a fixed position on the plunger 340 and the window 376 of the dose gauge 370 is in a fixed position on the syringe body 310, the position of the plunger 340 within the syringe body 310 at which one of the indicium 346 is visible via the window 376 corresponds to a volume (i.e., dosage volume) within the syringe body 310. As shown, the indicia 346 are non-volumetric indicia that correspond to a characteristic (i.e., the weight) of the patient. In other embodiments, the indicia 346 can correspond to any other characteristic of the patient, including the height, age, target body weight, and/or body mass index (BMI) of the patient. In other embodiments, the indicia 346 can correspond to a test result associated with the patient, including, for example, a range of blood sugar (e.g., for insulin dosage) or any other suitable test result. In this manner, a user can withdraw a dosage of medicament without the need for calculation or conversion to determine the volumetric amount. The series of indicia 346 also includes an arrow or alignment mark that can be aligned with the alignment protrusion 377 of the dose gauge 370.

The series of indicia 346 can be arranged along the outer surface 345 in any suitable manner or orientation. For example, in some embodiments, the series of indicia 346 is arranged coaxially along the longitudinal axis A_{L}. In this manner, when the plunger 340 is reciprocated along the longitudinal axis A_{L} within the internal volume 318 of the syringe body 310, each of the indicia 346 will be visible through the window 376 of the dose gauge 370 at a given plunger position.

As shown in FIG. 17, the plunger 340 includes an engagement protrusion (or rib) 348 that interfaces with the engagement slot 364 of the cap 360 to limit rotation of the plunger 340 about its longitudinal axis A_{L}. The engagement protrusion 348 extends from the proximal end portion 342 to the distal end portion 344 (and specifically to the point at which the elastomeric member 343 is coupled to the plunger 340). Thus, in use, the engagement protrusion 348 remains within the engagement slot 364 throughout the full range of motion of the plunger 340 during a delivery event. In particular, the engagement protrusion 348 remains within the engagement slot 364 when the plunger 340 moves between the first position (or dose ready position, see FIG. 21) and the second position (or dose delivered position, see FIGS. 8 and 9). Similarly stated, the engagement protrusion 348 is continuously within the engagement slot 364 when the plunger 340 moves between the first position and the second position.

Although shown as being a protrusion (i.e., rib or shoulder), in other embodiments, the plunger 340 can include any suitable engagement mechanism to engage with a corresponding portion of the cap 360 to limit rotation of the plunger 340 within the syringe body 310 about the longitudinal axis A_{L}. For example, in some embodiments, the plunger 340 can include a recessed portion (not shown) configured to receive a protrusion (not shown) of the cap 360. In other embodiments, the plunger 340 can include a splined surface that interfaces with a corresponding splined surface of the cap 360 to limit, reduce and/or prevent rotation of the plunger 340 within the syringe body 310.

In use, the distal end portion 344 of the plunger 340 can be reciprocated within the syringe body 310 along the longitudinal axis A_{L} to convey a medicament into and/or out of the internal volume 318 of the syringe body 310. In the initial configuration (see FIGS. 8 and 9), the plunger 340 is positioned at its distal-most position within the syringe body 310 (i.e., in the second position). In the second position, the distal surface 349 of the elastomeric member 343 is in contact with the corresponding internal tapered surface 334 of the syringe body 310. To prepare a dose of the medicament for delivery, the delivery tip 332 is placed in fluid communication with a source of medicament (e.g., a medicament container, not shown), and the plunger 340 is moved proximally, as shown by the arrow FF in FIG. 21. The movement of the distal end portion 344 of the plunger 340 within the syringe body 310 increases the internal volume 318, which, in turn, produces a vacuum that draws the medicament into the syringe body 310. As shown in FIG. 21, the plunger 340 is moved proximally until an indicium 346 (the indicium corresponding to a weight of 36-47 lb.) is visible through the window 376 of the dose gauge 370. Further, the alignment protrusion 377 of the dose gauge 370 is aligned with the alignment mark or arrow of the indicium. In this manner, the syringe assembly 300 is placed in its second (or “dosage set”) configuration (and the plunger 340 is in a first position within the syringe body 310).

As described above, during the movement of the plunger 340 from the second position to the first position, the engagement protrusion 348 of the plunger 340 interfaces with the engagement slot 364 of the cap 360 to limit rotation of the plunger 340 about the longitudinal axis A_{L} of the plunger. In this manner, the indicia 346 remain radially aligned with the window 376 of the dose gauge 370, thus ensuring that the indicia 346 will be visible through the window 376 of the dose gauge 370 when the plunger 340 is in the corresponding longitudinal position within the syringe body 310.

To deliver the dosage withdrawn, the user then places the delivery tip 332 in the desired location (e.g., in the patient's mouth) and moves the plunger 340 distally (i.e., opposite the direction DD shown in FIG. 21). The movement of the distal end portion 344 of the plunger 340 within the syringe body 310 decreases the internal volume 318, which, in turn, produces a pressure that conveys the medicament out of the syringe body 310. The plunger 340 is moved distally until the surface 349 of the elastomeric member 343 is in contact with the internal tapered surface 334 of the syringe body 310. This movement places the syringe assembly 300 in its third (or dose delivered) configuration. During the movement of the plunger 340 from the first position back to the second position, the engagement protrusion 348 of the plunger 340 interfaces with the engagement slot 364 of the cap 360 to limit rotation of the plunger 340 about the longitudinal axis A_{L} of the plunger.

In some embodiments, the syringe assembly 300 can be disassembled for cleaning or the like. Specifically, the plunger 340 can be removed from the syringe body 310 by first moving the plunger 340 proximally along the longitudinal axis A_{L} until the distal end portion 344 and/or the elastomeric member 343 passes through the opening 343 and the opening 368. At this point, the engagement protrusion 348 remains within the engagement slot 364, and the elastomeric member 343 is within the cap 360, between the distal surface 365 and the proximal surface 363. Further proximal movement along the longitudinal axis A_{L} is limited by contact between portions of the plunger 340 and the proximal surface 363 of the cap 360. Next, the distal end portion 344 of the plunger (including the elastomeric member 343) is moved through the lateral opening 367. This movement occurs in a direction nonparallel to the along the longitudinal axis A_{L}.

After cleaning, to prepare the syringe assembly 300 for use, the distal end portion 344 of the plunger 340 is placed within the volume 318 of the syringe body 310. Specifically, the distal end portion 344 is first moved through the lateral opening 367. The direction of motion of the distal end portion 344 is nonparallel to the longitudinal axis A_{L}. After the distal end portion 344 is through the lateral opening 367, it is then moved distally through the opening 368 and the opening 323, and into the volume 318. The direction of motion of the distal end portion 344 through the opening 368 is parallel to the longitudinal axis A_{L}. Thus, the movement of the distal end portion 344 occurs in two discrete motions. The distal end portion 344 of the plunger 340 can then be reciprocated within the syringe body 310 along the longitudinal axis A_{L}, as described above, to convey a medicament into and/or out of the internal volume 318 of the syringe body 310.

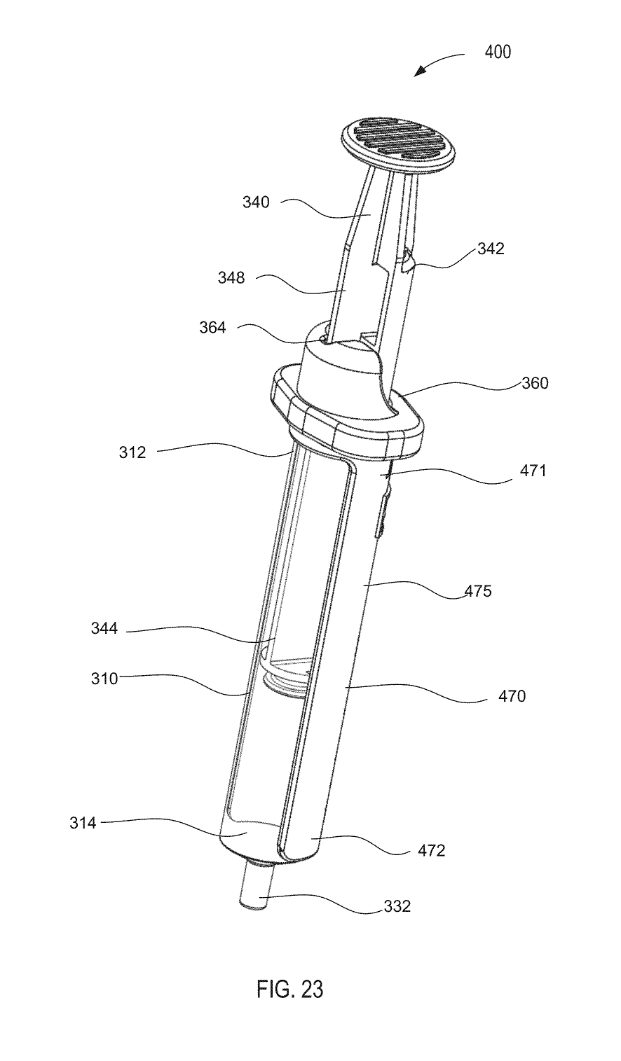

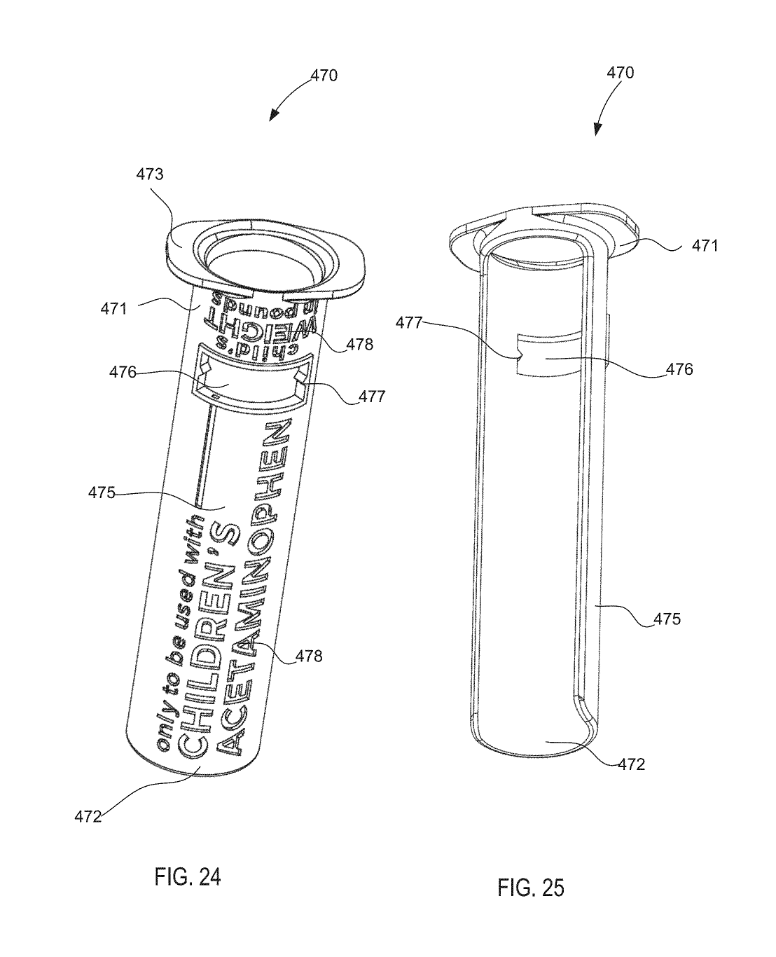

Although the dose gauge 370 is shown as extending only a portion of the length of the syringe body 310, in other embodiments, the dose gauge 370 (or any of the dose gauges described herein) can have any suitable length. For example, in some embodiments, a dose gauge can extend substantially the full length of the syringe body. For example, FIGS. 22-25 show various views of a syringe assembly 400, according to an embodiment. FIGS. 22 and 23 are a front perspective view and a back perspective view, respectively, of the syringe assembly 400 with the plunger 340 in a first position (i.e., dose readied) within the syringe body 310. The syringe assembly 400 includes a syringe body 310, a dose gauge 470, a cap 360, and a plunger 340. The syringe body 310 is the same as the syringe body 310 described above with reference to the syringe assembly 300, and is therefore not described in detail below. The cap 360 is the same as the cap 360 described above with reference to the syringe assembly 300, and is therefore not described in detail below. The plunger 340 is the same as the plunger 340 described above with reference to the syringe assembly 300, and is therefore not described in detail below. Thus, the syringe assembly 400 differs from the syringe assembly 300 in that the dose gauge 470 of the syringe assembly 400 is longer than, and covers more of the syringe body than the dose gauge 370.

The dose gauge 470 includes a proximal end portion 471 and a distal end portion 472, and is coupled about at least a portion of the side wall 415 of the syringe body 410. As shown, the dose gauge 470 defines a window 476 through which portions of the plunger 340 (i.e., the indicia 346) within the syringe body 310 can be seen (i.e., via a transparent portion of the side wall 315 of the syringe body 310). In this manner, the dose gauge 470 and the plunger 340 can provide a visual indication of the dosage of medicament drawn into the syringe body 310.

The dose gauge 470 includes an opaque portion 475 surrounding the window 476. In this manner, the opaque portion 475 can provide a frame that surrounds the window 476. Thus, during use, the indicia 346 displayed within the window 476 are clearly accentuated to the user. Moreover, the opaque portion 475 surrounding the window 476 includes at least one alignment protrusion 477. As shown in FIG. 22, the alignment protrusion 477 is configured to be aligned with the one indicium 346 from the series of indicia when the plunger 340 is in the first (dose ready) position. The outer surface of the dose gauge 470 can include instructions or indicia 478 associated with the medicament, dose, and/or treatment regimen. Such instructions or indicia can include, for example, the characteristic of the patient to be considered when setting the dosage (e.g., “child's weight”), the units of measure (e.g., pounds), alignment marks or arrows, identification of the regimen (e.g., “day 2”), and/or the drug name (e.g., “acetaminophen”).

The window 476 can be a portion of the dose gauge that is devoid of material. In other embodiments, however, the window 476 can include a transparent material through which a user can view the indicia 346. Moreover, the window 476 can be of any suitable size and/or shape. Thus, although shown as being rectangular, in other embodiments, the window 476 can be circular, oval, or any other polygon. Although the window 476 is shown as being sized such that only one indicium 446 from the series of indicia is visible through the window at a time, in other embodiments, the dose gauge 470 (or any of the dose gauges described herein) can include a window through which multiple indicia can be viewed at a time. In yet other embodiments, the dose gauge 470 (or any of the dose gauges described herein) can include more than one window.

The proximal end portion 471 of the dose gauge 470 includes a flange that has a top surface 473. The top surface 473 is configured to engage the distal surface 326 of the syringe flange 322 to couple the dose gauge 470 to the flange 322. In this manner, the dose gauge 470 is indexed to the flange 322 and/or the distal surface 326. By indexing the dose gauge 470 to the flange 322 and/or the syringe body 310, the dose gauge 470 can be maintained in a fixed location along the longitudinal axis A_{L} of the syringe body 310, thereby producing an accurate and repeatable indication of the dose amount when the indicia 346 of the plunger 340 are aligned within the window 476.

The dose gauge 470 can be coupled to the syringe body 410 in any suitable manner. In some embodiments, the top surface 473 can be bonded to the flange 322 of the syringe body 310 (e.g., via an adhesive, a weld joint or the like). In other embodiments, the dose gauge 470 can be coupled to the cap 360 in a manner that surrounds the flange 322 of the syringe body 310 to couple the dose gauge 470 to the syringe body. In yet other embodiments, the dose gauge 470 is snapped into place over a portion of the syringe body 310 and/or the cap 360 (i.e., via an interference fit).

Referring to FIG. 23, the opaque portion 475 extends around approximately one half of the circumference of the syringe body 310 (about 270 degrees). By exposing certain portion (e.g., the “back side) of the syringe body 310, the user can visually inspect the medicament in the syringe body 310 for air bubbles, the correct color of the medicament, and the like. In other embodiments, the dose gauge 470 (or any of the dose gauges described herein) can extend around any suitable portion of the circumference of the syringe body 410.

As shown in FIG. 23, the dose gauge 470 has a length that is substantially the same as the length of the syringe body 310 (e.g., from the flange 322 to the beginning of the tapered surface 334). In this manner, the dose gauge 470 covers the entire length of the syringe body 310, thereby blocking the view of the plunger indicia 346 unless the indicia is located within the window 476. In this manner, the extended length dose gauge 470 can reduce the likelihood that the user will inadvertently use indicia 346 that are not within the window to set the dosage. Moreover, the extended length also allows for more room on which to include the indicia 478.

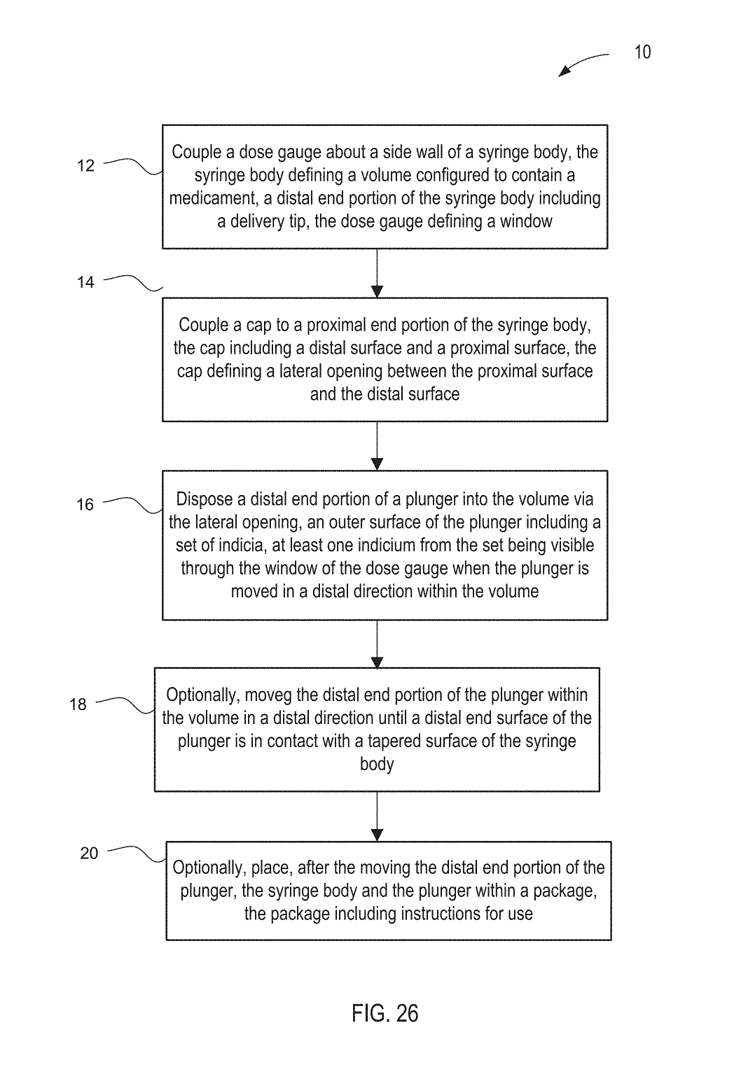

FIG. 26 is a flow chart illustrating a method 10 of producing (or assembling) a syringe assembly, according to an embodiment. The method 10 can be performed using any of the syringe assemblies or components shown and described herein. The method includes coupling a dose gauge about a side wall of a syringe body, at 12. The syringe body, which can be any of the syringe bodies disclosed herein (including the syringe body 310), defines a volume (e.g., volume 318) configured to contain a medicament. A distal end portion of the syringe body includes a delivery tip (e.g., the delivery tip 332). The dose gauge, which can be any of the dose gauges disclosed herein (including the dose gauge 370 or the dose gauge 470), defines a window.

The dose gauge can be coupled to the syringe body in any suitable manner. For example, in some embodiments, the dose gauge can be permanently (or non-removably) coupled to the syringe body via an adhesive, a weld joint or the like. In other embodiments, the dose gauge can be coupled to and/or about the syringe assembly via an interference fit (e.g., by snapping the dose gauge about the outer surface of the syringe body). In yet other embodiments, a proximal end portion of the syringe body can include a flange (e.g., the flange 322), and the dose gauge can be coupled to a distal surface of the flange (e.g., via an epoxy).

A cap is coupled to a proximal end portion of the syringe body, at 14. The cap, which can be any of the caps disclosed herein (including the cap 360), includes a distal surface and a proximal surface, and defines a lateral opening between the proximal surface and the distal surface (e.g., the lateral opening 376 and/or the lateral opening 476). The cap can be coupled to the syringe body in any suitable manner. For example, in some embodiments, the cap can be permanently (or non-removably) coupled to the syringe body via an adhesive, a weld joint or the like. In other embodiments, the cap can be coupled to and/or about the syringe assembly via an interference fit (e.g., by snapping the dose gauge about the outer surface of the syringe body and/or the dose gauge). In yet other embodiments, a proximal end portion of the syringe body can include a flange (e.g., the flange 322), and the cap can be coupled to a proximal surface of the flange (e.g., via an epoxy). Moreover, in some embodiments, the cap and the dose gauge can be monolithically constructed and can be coupled to the syringe body together in a single operation.

The method further includes disposing a distal end portion of a plunger into the volume via the lateral opening, at 16. The plunger, which can be any of the plungers described herein (e.g., the plunger 340) includes an outer surface having a series of indicia (e.g., the indicia 346). At least one indicium is visible through the window of the dose gauge when the plunger is moved in a distal direction within the volume of the syringe body. The distal end portion of the plunger can be disposed within the volume by any suitable series of steps. For example, in some embodiments, the distal end portion of the plunger can be first moved in a lateral direction through the lateral opening of the cap, and then moved at a second time through a top opening (e.g., the opening 323) defined by the syringe body. The lateral direction being nonparallel to the distal direction. In some embodiments, an engagement portion of the plunger is first aligned with a corresponding engagement portion of the cap to allow the plunger to be disposed within the volume.

In some embodiments, the method optionally includes moving the distal end portion of the plunger within the volume in a distal direction until a distal end surface of the plunger is in contact with a tapered surface of the syringe body (e.g., an internal tapered surface 334), at 18. In this manner, the plunger can be placed in the second position.

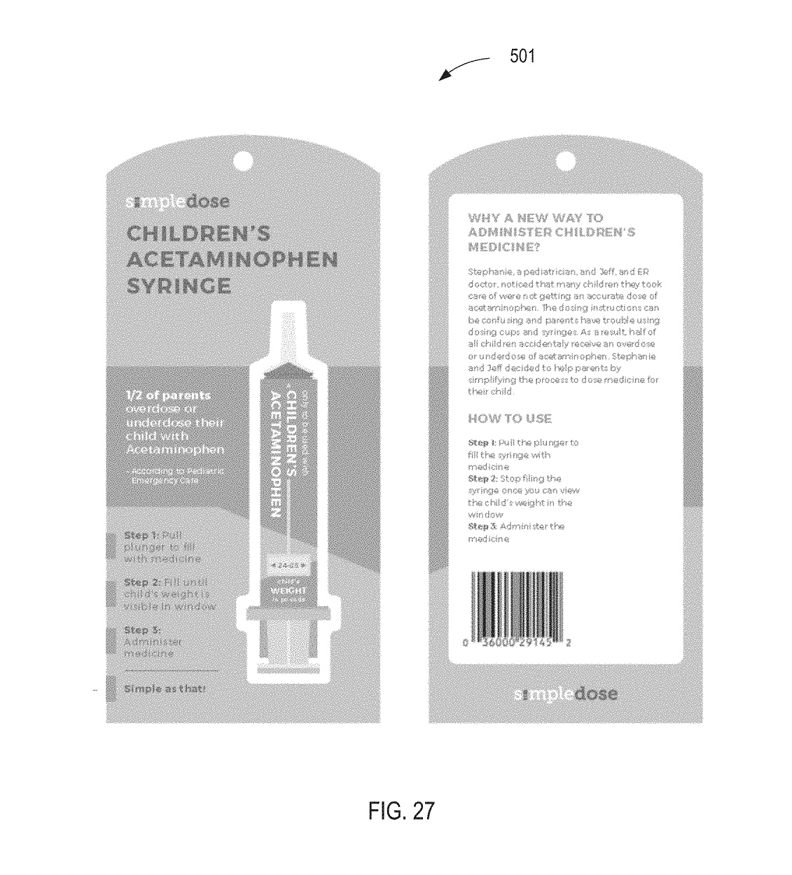

In some embodiments, the method optionally includes placing the syringe body and the plunger within a package, at 20. The package can be any suitable package, such as the package 501 shown in FIG. 27, and can include instructions for use and other information.

The syringe body 110 and any of the syringe bodies shown and described herein can be constructed of any suitable material, such as hard plastic or glass. In some embodiments, the syringe body 110, or any of the syringe bodies described herein, can be constructed from moldable plastic materials such as, for example, a polymeric plastic including, but not limited to, polyethylene, polypropylene, polycarbonate, polytetrafluoroethylene (PTFE), a phenol formaldehyde resin (e.g., Bakelite) and/or the like. In some embodiments, the syringe body 310, or any of the syringe bodies shown and described herein, is monolithically constructed. For example, the syringe body 310 and any of the syringe bodies shown and described herein can be molded to form a single component having a constant cross-sectional diameter. In other embodiments, however, the syringe body 310, and any of the syringe bodies shown and described herein, can be constructed from multiple separate components that are later joined together.

The plunger 340 and any of the plungers shown and described herein can be constructed of any suitable material, such as hard plastic. In some embodiments, the plunger 340, or any of the plungers described herein, can be constructed from moldable plastic materials such as, for example, a polymeric plastic including, but not limited to, polyethylene, polypropylene, polycarbonate, polytetrafluoroethylene (PTFE), a phenol formaldehyde resin (e.g., Bakelite) and/or the like. In some embodiments, the plunger 340, or any of the plungers shown and described herein, is monolithically constructed. In other embodiments, however, the plunger 340, and any of the plungers shown and described herein, can be constructed from multiple separate components that are later joined together. For example, in some embodiments, the plunger 340 can include a hard plastic member and an elastomeric member that seals the medicament within the internal volume of the accompanying syringe body. In such embodiments, the elastomeric member can be of any design or formulation suitable for contact with the medicament.

In some embodiments, a syringe body can be an off-the-shelf syringe with a cap and a dose gauge glued to the existing syringe body. As described above, the cap fits over the flange of the syringe body and hangs over the edge of the flange of the syringe body. The dose gauge sits underneath the overhang of the flange of the syringe body and adheres to the underside of the flange of the syringe body. An epoxy can attach all three components.

In some embodiments, a syringe assembly can be used in conjunction with different medicaments. Similarly stated, in some embodiments, a syringe assembly can include multiple sets of indicia, each of which corresponds to a different “scale” or dosage setting for a different drug. In some embodiments, the delivery tip can be a protrusion extending from the syringe body that can be received within a medicament container (e.g., bottle, vial or the like) and that can also deliver the medicament orally to the patient. In other embodiments, the delivery tip can a tapered fitting (such as a Luer fitting) that is adapted to couple the distal end portion of the syringe body to a needle (not shown).

In some embodiments, any of the syringe assemblies described herein can be included within a kit that also contains a medicament container, a delivery member (e.g. a needle, an oral delivery tip, or the like) associated packaging and/or an instruction set.

In some embodiments, a kit can include features to reduce the likelihood that the syringe assembly therein will be used with a drug for which the assembly is not intended. For example, in some embodiments, a kit can include a coupling member that retains the syringe assembly to the medicament container.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

For example, any of the syringe assemblies described herein can include any suitable engagement mechanism (or mating engagement portions) to limit rotation of the plunger within the syringe body. In some embodiments, such engagement portions can include, for example, a protrusion configured to be disposed within a corresponding groove of the plunger. In other embodiments, such engagement portions can include a recessed portion configured to receive a protrusion of the plunger. In yet other embodiments, such engagement portions can include a splined surface that interfaces with a corresponding splined surface of the plunger to limit, reduce and/or prevent rotation of the plunger within the syringe body.

Any of the windows of the dose gauge shown and described herein can be of any suitable size and/or shape to allow visual access to the indicia. For example, as shown, the window can be defined by a side wall that includes arrows, pointers or other features to enhance the longitudinal alignment between the plunger and the window.

Although the dose gauge 370 is shown and described as being a separate member, in other embodiments, a dose gauge can be an opaque label coupled to the side wall of the syringe body. In such embodiments, the opaque label can define the window such that the opaque label frames the window.

Although the syringe body 110 and the cap 160 are shown as having a substantially circular cross-sectional shape, in other embodiments, any of the syringe bodies, plungers or other components described herein can have any suitable shape. For example, in some embodiments, a plunger and/or a syringe body can have a non-circular cross-sectional area to limit rotation of the plunger within the syringe body (i.e., about its longitudinal axis).

Any of the plungers shown and described herein can include an elastomeric member, such as the elastomeric member 343 shown and described above. Any such elastomeric member can be constructed from any suitable material, and can be formulated to be compatible with the medicament housed within the syringe body. Similarly stated, the elastomeric member can be formulated to minimize any reduction in the efficacy of the medicament that may result from contact (either direct or indirect) between the elastomeric member and the medicament. In some embodiments, at least a portion of the elastomeric member can be coated to improve the lubricity of the elastomeric member.

In some embodiments, the side wall of the syringe body 310 (or any of the syringe bodies shown herein) or the cap 360 (or any of the caps shown herein) can include a syringe detent that engages a portion of the plunger within the syringe body when each of the series of indicia (e.g., the indicia 346) is visible through the dose gauge. In this manner, the user can receive a tactile sensation (e.g., a slight snap, click or vibration) when each indicium is aligned with and/or visible via the window. In this manner, the syringe assembly can allow for a series of discrete intervals of plunger movement within the syringe body.

Although the plunger 140 is shown as having only two indicia 146, in other embodiments, the plunger 140 (or any of the plungers described herein) can include any number of indicia. For example, in some embodiments, the plunger 140 (or any of the plungers described herein) can include a series of indicia corresponding to the following weight ranges of the patient: 12 to 17 pounds, 18 to 23 pounds, 24 to 35 pounds, 36 to 47 pounds, 48 to 60 pounds, 61 to 75 pounds, 76 to 90 pounds, and greater than 90 pounds. In other embodiments, the plunger 140 (or any of the plungers described herein) can include a series of indicia corresponding to the following age ranges of the patient: 6 to 18 months, 18 months to 3 years, 3 to 5 years, 6 to 11 years, and 12 years and higher. In yet other embodiments, the plunger 140 (or any of the plungers described herein) can include a series of indicia that do not correspond to ranges (e.g., they may correspond to a specific weight, age, height, lab test result or the like). Such indicia may include for example, a metric/English marking (10 lb/4.5 kg).

Although the plunger 340 is shown as having a series of indicia 346 arranged longitudinally along the plunger 340, in other embodiments, the plunger 340 (and any of the plungers described herein) can also include an indicium (e.g., an instruction indicia, a warning or the like) on the proximal end portion that is exposed from the syringe body when the syringe assembly is in its first configuration. For example, in some embodiments, the plunger 340 (and any of the plungers described herein can include an indicium facing outwardly from an activation surface or “handle.”

Although the syringe assemblies are shown and described herein as being suitable for multiple uses (e.g., for a therapeutic regimen over several days and/or doses), in other embodiments, any of the syringe assemblies described herein can be configured for a single-use application. In this manner, the likelihood of using the syringe assembly with the wrong medicament is reduced. For example, in some embodiments, a syringe assembly can include a locking tab (e.g., on a flange of the syringe body) that engages and retains the plunger in the distal-most position after a single dose has been delivered. In other embodiments, the syringe assembly can include a frangible portion that breaks and/or permanently deforms such that the plunger cannot be repeatedly reciprocated within the syringe body.

In some embodiments, a syringe body includes a lock member configured to limit movement of the distal end portion of the plunger in a proximal direction within the syringe body after a dose of the medicament has been delivered from the volume of the syringe body.

In some embodiments, a syringe body includes a protrusion configured to engage a portion of the plunger to limit movement of the distal end portion of the plunger in a proximal direction within the syringe body when the distal end portion of the plunger is in a distal-most position within the syringe body.

Any of the devices and/or medicament containers shown and described herein can be constructed from any suitable material. Such materials include glass, plastic (including thermoplastics such as cyclic olefin copolymers), or any other material used in the manufacture of syringes containing medications.

In some embodiments, a syringe body and a cap can be monolithically constructed so that the two components are formed in one process (e.g., injection molding). A dose gauge can be painted on the injection molded component to make it opaque. In another embodiment, a dose gauge can be a decal, which is then placed on the injection molded component. In yet another embodiment, a dose gauge can be a plastic add-on component, which can be glued to the injection molded component.

In some embodiments, a syringe body and a dose gauge can be monolithically constructed so that the two components are formed in one process. A cap can be a plastic add-on component, which can be glued to the syringe body / dose gauge.

In some embodiments, a dose gauge is designed for one specific medicine and can be attached to a general over the counter syringe. For example, in some embodiments, the dose gauge is designed for ibuprofen or acetaminophen.

In some embodiments, a dose gauge and a cap are monolithically constructed and are attached on the syringe body as one component.

In some embodiments, a cap is removable from the syringe body and is of various shapes such as a removable star, Spiderman or other superheroes, princess, animal shapes, cartoon characters, etc. The ability to customize the shape of the cap provides a more palatable manner to offer medicine to children.

Any of the devices and/or medicament containers shown and described herein can include and/or be used with any suitable medicament or therapeutic agent. In some embodiments, the medicament contained within any of the medicament containers and/or syringe assemblies shown herein can include acetaminophen, ibuprofen, diphenhydramine (Benadryl), cough and cold medicines, vitamins, Prescriptions: azithromycin, albuterol, allopurinol, cefdinir, chloroquine, choleystyramine, doxycycline, enoxaparin, erythromycin, hydroxychloroquine, isoniazid, levofloxacin, magnesium sulfate, methadone, omeprazole, Tamiflu, roxicet, pyridium, Compazinem Phenergan, kayexelate, prednisone, prednisolone, and/or dexamethasone.

The syringe assemblies disclosed herein can contain any suitable amount of any medicament. For example, in some embodiments, a syringe assembly as shown herein can be sized and/or can define a volume sufficient to contain any suitable dosage of medicament.

Although various embodiments have been described as having particular features and/or combinations of components, other embodiments are possible having a combination of any features and/or components from any of embodiments where appropriate. For example, any of the syringe assemblies described herein can include a syringe detent configured to engage one or more plunger detents to resist movement of the plunger within the syringe body when each of a series of indicia is visible through a window of the dose gauge. In this manner, the user can receive a tactile sensation (e.g., a slight snap, click or vibration) when each of the indicia are aligned with and/or visible via the window. In this manner, the syringe assembly can allow for a series of discrete intervals of plunger movement within the syringe body.

Any of the kits described herein can be used with and/or include any of the syringe assemblies shown and described herein.

1. An apparatus, comprising:
a syringe body defining a volume configured to contain a medicament, a distal end portion of the syringe body including a delivery tip;
a dose gauge disposed about at least a portion of a side wall of the syringe body, the dose gauge defining a window;
a cap coupled to a proximal end portion of the syringe body, the cap including an engagement portion; and
a plunger having a distal end portion configured to move within the volume of the syringe body from a first position to a second position to convey the medicament, an engagement portion of the plunger configured to interface with the engagement portion of the cap to prevent rotation of the plunger about a longitudinal axis of the plunger, an outer surface of the plunger including a plurality of indicia, at least one indicium from the plurality of indicia being visible through the window of the dose gauge when the plunger is in the first position.

2. (canceled)

3. (canceled)

4. The apparatus of claim 1, wherein:
the proximal end portion of the syringe body includes a flange; and
the dose gauge and the cap are coupled about the flange.

5. The apparatus of claim 1, wherein:
a proximal surface of the cap includes the engagement portion;
a distal surface of the cap is coupled to at least one of the proximal end portion of the syringe body or the dose gauge; and
the cap defines a lateral opening between the proximal surface and the distal surface, the lateral opening sized such that the distal end portion of the plunger can be removed from the volume of the syringe body via the lateral opening.

6. The apparatus of claim 5, wherein:
the distal end portion of the plunger includes an elastomeric member defining a fluid-tight seal with the side wall of the syringe body; and
a height of the lateral opening is greater than a height of the elastomeric member, a width of the lateral opening is greater than a diameter of the elastomeric member.

7. The apparatus of claim 1, wherein:
the engagement portion of the cap defines a slot; and
the engagement portion of the plunger includes a protrusion that extends along the longitudinal axis of the plunger, the protrusion disposed within the slot.

8. The apparatus of claim 7, wherein:
the distal end portion of the syringe body includes a tapered surface; and
a distal end surface of the plunger is in contact with the tapered surface when the plunger is in the second position, the protrusion of the plunger being continuously within the slot when the plunger moves from the first position to the second position.

9. The apparatus of claim 1, wherein the dose gauge and the cap are monolithically constructed.

10.-11. (canceled)

12. The apparatus of claim 1, wherein:
the proximal end portion of the syringe body includes a flange; and
a proximal surface of the dose gauge is coupled to a surface of the flange, a distal end surface of the dose gauge extends to the distal end portion of the syringe body.

13. The apparatus of claim 1, wherein:
the window is an opening defined by an opaque side wall of the dose gauge, the side wall of the dose gauge covering a first portion of a circumference of the syringe body such that the medicament within the volume when the plunger is in the first position can be viewed through a second portion of the syringe body, the second portion being transparent.

14. (canceled)

15. The apparatus of claim 10, wherein:
the plurality of indicia is a plurality of dose indicia arranged coaxially along the longitudinal axis of the plunger and at a constant circumferential position along the outer surface of the plunger such that each of the plurality of indicia is visible through the window when the plunger moves within the volume of the syringe body from the first position to the second position; and
the engagement portion of the plunger is continuously engaged with the engagement portion of the cap when the plunger moves from the first position to the second position.

16. An apparatus, comprising:
a syringe body defining a volume configured to contain a medicament, a distal end portion of the syringe body including a delivery tip, a proximal end portion of the syringe body defining a first opening through which the volume can be accessed, the proximal end portion of the syringe body including a cap having a distal surface and a proximal surface, the proximal surface extending above the first opening, the cap defining a second opening between the proximal surface and the distal surface, a central axis of the second opening being nonparallel to a central axis of the first opening, the syringe body including a dose gauge disposed about a side wall of the syringe body, the dose gauge defining a window; and
a plunger having a distal end portion configured to move within the volume of the syringe body from a first position to a second position to convey the medicament, an outer surface of the plunger including a plurality of indicia, at least one indicium from the plurality of indicia being visible through the window of the dose gauge when the plunger is in the first position, the second opening sized such that the distal end portion of the plunger can be removed from the volume of the syringe body via the second opening.

17. The apparatus of claim 16, wherein:
the distal end portion of the plunger includes an elastomeric member defining a fluid-tight seal with the side wall of the syringe body; and
a height of the second opening is greater than a height of the elastomeric member; a width of the lateral second is greater than a diameter of the elastomeric member.

18. (canceled)

19. The apparatus of claim 16, wherein:
the proximal surface of the cap defines a slot; and
the plunger includes a protrusion that extends along the longitudinal axis of the plunger, the protrusion disposed within the slot to prevent rotation of the plunger about a longitudinal axis of the plunger when the plunger is moved from the first position to the second position.

20. The apparatus of claim 19, wherein:
the distal end portion of the syringe body includes a tapered surface; and
a distal end surface of the plunger is in contact with the tapered surface when the plunger is in the second position, the protrusion of the plunger being continuously within the slot when the plunger moves from the first position to the second position.

21.-22. (canceled)

23. The apparatus of claim 16, wherein:
the proximal end portion of the syringe body includes a flange;
the dose gauge is coupled to a first surface of the flange; and
the cap is coupled to a second surface of the flange.

24. (canceled)

25. A method, comprising:
coupling a dose gauge about a side wall of a syringe body, the syringe body defining a volume configured to contain a medicament, a distal end portion of the syringe body including a delivery tip, the dose gauge defining a window;
coupling a cap to a proximal end portion of the syringe body, the cap including a distal surface and a proximal surface, the cap defining a lateral opening between the proximal surface and the distal surface; and
disposing a distal end portion of a plunger into the volume via the lateral opening, an outer surface of the plunger including a plurality of indicia, at least one indicium from the plurality of indicia being visible through the window of the dose gauge when the plunger is moved in a distal direction within the volume.

26. The method of claim 25, wherein the disposing includes:
moving, at a first time, the distal end portion of the plunger in a lateral direction through the lateral opening; and
moving, at a second time after the first time, the distal end portion of the plunger in the distal direction through a top opening defined by the syringe body, the lateral direction being nonparallel to the distal direction.

27. The method of claim 25, wherein the disposing includes:
moving, after the aligning, the distal end portion of the plunger in a lateral direction through the lateral opening; and
moving the distal end portion of the plunger in the distal direction through a top opening defined by the syringe body, the lateral direction being nonparallel to the distal direction, the engagement portion of the cap interfacing with the engagement portion of the plunger during the moving the distal end portion of the plunger in the distal direction such that rotation of the plunger about a longitudinal axis of the plunger is prevented.

28. The method of claim 25, wherein:
the proximal end portion of the syringe body includes a flange; and
the coupling the dose gauge includes coupling a proximal surface of the dose gauge to a distal surface of the flange.

29.-30. (canceled)

31. The method of claim 25, wherein:
the distal end portion of the plunger includes an elastomeric member defining a fluid-tight seal with the side wall of the syringe body; and
a height of the lateral opening is greater than a height of the elastomeric member, a width of the lateral opening is greater than a diameter of the elastomeric member.

32. The method of claim 25, wherein the distal end portion of the syringe body includes a tapered surface, the method further comprising:
moving the distal end portion of the plunger within the volume in a distal direction until a distal end surface of the plunger is in contact with the tapered surface; and
placing, after the moving the distal end portion of the plunger, the syringe body and the plunger within a package, the package including instructions for use.
